(19) United States
(12) Patent Application Publication
(10) Pub. No.: US 2010/0004245 A1
(43) Pub. Date: Jan. 7, 2010

(54) AZACYCLOALKANE DERIVATIVES AS INHIBITORS OF STEAROYL-COENZYME A DELTA-9 DESATURASE

(75) Inventors: Renata M. Oballa, Kirkland (CA); Denis Deschenes, Dorval (CA); Marc Gagnon, Montreal (CA); Yves Leblanc, Kirkland (CA); David Powell, Verdun (CA); Yeeman K. Ramtohul, Pierrefonds (CA)

Correspondence Address: MERCK AND CO., INC, P O BOX 2000, RAHWAY, NJ, 07065-0907, US

(73) Assignee: MERCK FROSST CANADA LTD., Kirkland, QC (CA)

(21) Appl. No.: 12/440,835

(22) PCT Filed: Oct. 18, 2007
(86) PCT No.: PCT/CA2007/001858

§ 371 Date: Mar. 11, 2009

Related U.S. Application Data
(60) Provisional application No. 60/853,199, filed on Oct. 20, 2006.

(51) Int. Cl. A61K 31/53; C07D 401/14; C07D 403/14; C07D 417/14; C07D 413/14; A61K 31/4545; A61K 31/501; A61K 31/506

(52) U.S. Cl. 514/242; 546/193; 544/238; 544/182; 514/318; 514/252.02; 514/256

(57) ABSTRACT

Azacycloalkane derivatives of structural formula (I) are selective inhibitors of stearoyl-coenzyme A delta-9 desaturase (SCD1) relative to other known stearoyl-coenzyme A desaturases. The compounds of the present invention are useful for the prevention and treatment of conditions related to abnormal lipid synthesis and metabolism, including cardiovascular disease, such as atherosclerosis; obesity; diabetes; neurological disease; metabolic syndrome; insulin resistance; and liver steatosis.

### FIELD OF THE INVENTION

The present invention relates to azacycloalkane derivatives which are inhibitors of stearoyl-coenzyme A delta-9 desaturase (SCD) and the use of such compounds to control, prevent and/or treat conditions or diseases mediated by SCD activity. The compounds of the present invention are useful for the control, prevention and treatment of conditions and diseases related to abnormal lipid synthesis and metabolism, including cardiovascular disease, such as atherosclerosis; obesity; diabetes; neurological disease; metabolic syndrome; insulin resistance; cancer; and hepatic steatosis.

### BACKGROUND OF THE INVENTION

At least three classes of fatty acyl-coenzyme A (CoA) desaturases (delta-5, delta-6 and delta-9 desaturases) are responsible for the formation of double bonds in mono- and polyunsaturated fatty acyl-CoAs derived from either dietary sources or de novo synthesis in mammals. The delta-9 specific stearoyl-CoA desaturases (SCDs) catalyze the rate-limiting formation of the cis-double bond at the C9-C10 position in monounsaturated fatty acyl-CoAs. The preferred substrates are stearoyl-CoA and palmitoyl-CoA, with the resulting oleoyl and palmitoleoyl-CoA as the main components in the biosynthesis of phospholipids, triglycerides, cholesterol esters and wax esters (Dobrzyn and Natami, Obesity Reviews, 6: 169-174 (2005)).

The rat liver microsomal SCD protein was first isolated and characterized in 1974 (Strittmatter et al., PNAS 71: 4565-4569 (1974)). A number of mammalian SCD genes have since been cloned and studied from various species. For example, two genes have been identified from rat (SCD1 and SCD2, Thiede et al., J. Biol. Chem., 261, 13230-13235 (1986)), Mihara, K., J. Biochem. (Tokyo), 108: 1022-1029 (1990)); four genes from mouse (SCD1, SCD2, SCD3 and SCD4) (Miyazaki et al., J. Biol. Chem., 278: 33904-33911 (2003)); and two genes from human (SCD1 and ACOD4 (SCD2)), (Zhang, et al., Biochem. J., 340: 255-264 (1991); Beiraghi, et al., Gene, 309: 11-21 (2003); Zhang et al., Biochem. J., 388: 135-142 (2005)). The involvement of SCDs in fatty acid metabolism has been known in rats and mice since the 1970's (Oshino, N., Arch. Biochem. Biophys., 149: 378-387 (1972)). This has been further supported by the biological studies of a) Asebia mice that carry the natural mutation in the SCD1 gene (Zheng et al., Nature Genetics, 23: 268-270 (1999)), b) SCD1-null mice from targeted gene deletion (Ntambi, et al., PNAS, 99: 11482-11486 (2002), and c) the suppression of SCD1 expression during leptin-induced weight loss (Cohen et al., Science, 297: 240-243 (2002)). The potential benefits of pharmacological inhibition of SCD activity has been demonstrated with anti-sense oligonucleotide inhibitors (ASO) in mice (Jiang, et al., J. Clin. Invest., 115: 1030-1038 (2005)). ASO inhibition of SCD activity reduced fatty acid synthesis and increased fatty acid oxidation in primary mouse hepatocytes. Treatment of mice with SCD-ASOs resulted in the prevention of diet-induced obesity, reduced body adiposity, hepatomegaly, steatosis, postprandial plasma insulin and glucose levels, reduced de novo fatty acid synthesis, decreased the expression of lipogenic genes, and increased the expression of genes promoting energy expenditure in liver and adipose tissues. Thus, SCD inhibition represents a novel therapeutic strategy in the treatment of obesity and related metabolic disorders.

There is compelling evidence to support that elevated SCD activity in humans is directly implicated in several common disease processes. For example, there is an elevated hepatic lipogenesis to triglyceride secretion in non-alcoholic fatty liver disease patients (Diraison, et al., Diabetes Metabolism, 29: 478-485 (2003)); Donnelly, et al., J. Clin. Invest., 115: 1343-1351 (2005)). The postprandial de novo lipogenesis is significantly elevated in obese subjects (Marques-Lopes, et al., American Journal of Clinical Nutrition, 73: 252-261 (2001)). There is a significant correlation between a high SCD activity and an increased cardiovascular risk profile including elevated plasma triglycerides, a high body mass index and reduced plasma HDL (Attie, et al., J. Lipid Res., 43: 1899-1907 (2002)). SCD activity plays a key role in controlling the proliferation and survival of human transformed cells (Scaglia and Igal, J. Biol. Chem., (2005)).

Other than the above mentioned anti-sense oligonucleotides, inhibitors of SCD activity include non-selective thia-fatty acid substrate analogs [B. Behrouzian and P. H. Buist, Prostaglandins, Leukotrienes, and Essential Fatty Acids, 68: 107-112 (2003)], cyclopropenoid fatty acids (Raju and Reiser, J. Biol. Chem., 242: 379-384 (1967)), certain conjugated long-chain fatty acid isomers (Park, et al., Biochim. Biophys. Acta, 1486: 285-292 (2000)), a series of pyridazine derivatives disclosed in published international patent application publications WO 2005/011653, WO 2005/011654, WO 2005/011656, WO 2005/011656, and WO 2005/011657, all assigned to Xenon Pharmaceuticals, Inc., and a series of heterocyclic derivatives disclosed international patent application publications WO 2006/014168, WO 2006/034279, WO 2006/034312, WO 2006/034315, WO 2006/034338, WO 2006/034341, WO 2006/034440, WO 2006/034441, and WO 2006/034446, all assigned to Xenon Pharmaceuticals, Inc.

The present invention is concerned with novel azacycloalkane derivatives as inhibitors of stearoyl-CoA delta-9 desaturase which are useful in the treatment and/or prevention of various conditions and diseases mediated by SCD activity including those related, but not limited, to elevated lipid levels, as exemplified in non-alcoholic fatty liver disease, cardiovascular disease, obesity, diabetes, metabolic syndrome, and insulin resistance.

The role of stearoyl-coenzyme A desaturase in lipid metabolism has been described by M. Miyazaki and J. M. Ntambi, Prostaglandins, Leukotrienes, and Essential Fatty Acids, 68: 113-121 (2003). The therapeutic potential of the pharmacological manipulation of SCD activity has been described by A. Dobryzn and J. M. Ntambi, in “Stearoyl-CoA desaturase as a new drug target for obesity treatment,” Obesity Reviews, 6: 169-174 (2005).

### SUMMARY OF THE INVENTION

The present invention relates to azacycloalkane derivatives of structural formula I:

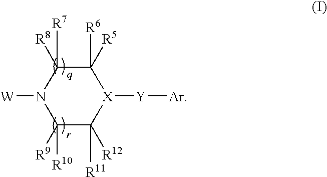

These azacycloalkane derivatives are effective as inhibitors of SCD. They are therefore useful for the treatment, control or prevention of disorders responsive to the inhibition of SCD, such as diabetes, insulin resistance, lipid disorders, obesity, atherosclerosis, and metabolic syndrome.

The present invention also relates to pharmaceutical compositions comprising the compounds of the present invention and a pharmaceutically acceptable carrier.

The present invention also relates to methods for the treatment, control, or prevention of disorders, diseases, or conditions responsive to inhibition of SCD in a subject in need thereof by administering the compounds and pharmaceutical compositions of the present invention.

The present invention also relates to methods for the treatment, control, or prevention of Type 2 diabetes, insulin resistance, obesity, lipid disorders, atherosclerosis, and metabolic syndrome by administering the compounds and pharmaceutical compositions of the present invention.

The present invention also relates to methods for the treatment, control, or prevention of obesity by administering the compounds of the present invention in combination with a therapeutically effective amount of another agent known to be useful to treat the condition.

The present invention also relates to methods for the treatment, control, or prevention of Type 2 diabetes by administering the compounds of the present invention in combination with a therapeutically effective amount of another agent known to be useful to treat the condition.

The present invention also relates to methods for the treatment, control, or prevention of atherosclerosis by administering the compounds of the present invention in combination with a therapeutically effective amount of another agent known to be useful to treat the condition.

The present invention also relates to methods for the treatment, control, or prevention of lipid disorders by administering the compounds of the present invention in combination with a therapeutically effective amount of another agent known to be useful to treat the condition.

The present invention also relates to methods for treating metabolic syndrome by administering the compounds of the present invention in combination with a therapeutically effective amount of another agent known to be useful to treat the condition.

### DETAILED DESCRIPTION OF THE INVENTION

The present invention is concerned with azacycloalkane derivatives useful as inhibitors of SCD. Compounds of the present invention are described by structural formula I:

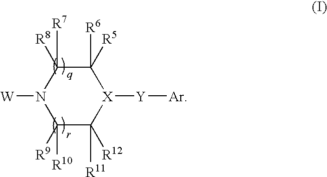

and pharmaceutically acceptable salts thereof; wherein
each m is independently an integer from 0 to 4;
each n is independently an integer from 0 to 2;
each s is independently an integer from 1 to 3;
each t is independently an integer from 1 to 3;
q is 0 or 1;
r is 0 or 1;
Z is O, S, or NR⁴;
X—Y is N—C(O), N—CR^{a}R^{b}, CR¹⁴—O, CR¹⁴—S(O)₀₋₂, or CR¹³—CR^{a}R^{b};
W is heteroaryl selected from the group consisting of:

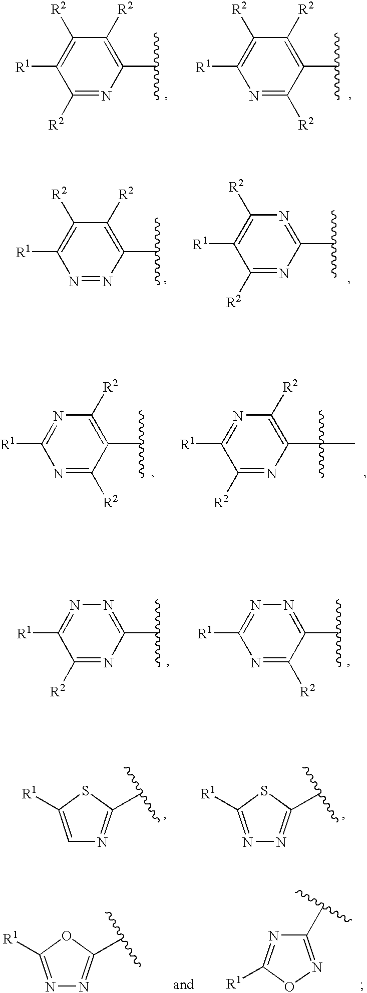

Ar is phenyl, naphthyl, or heteroaryl optionally substituted with one to five R³ substituents;
R^{a} and R^{b} are each independently hydrogen or C₁₋₃ alkyl, wherein alkyl is optionally substituted with one to three substituents independently selected from fluorine and hydroxy;
R¹ is heteroaryl selected from the group consisting of:

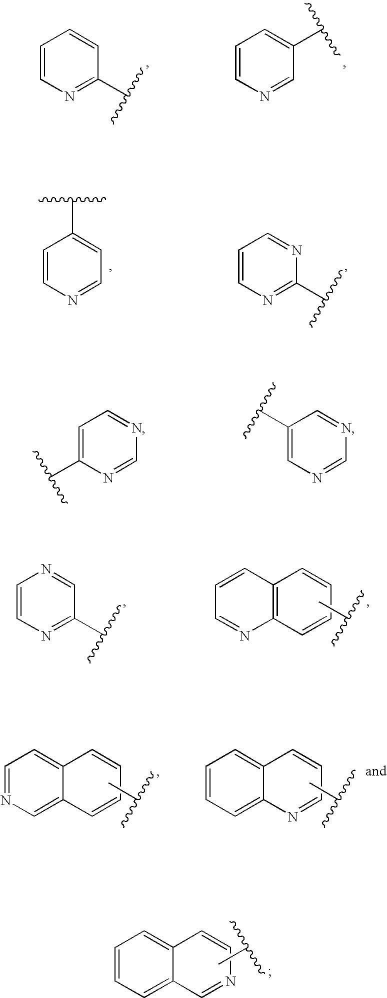

wherein heteroaryl is monosubstituted with —(CH₂)ₘCO₂H or —(CH₂)ₘCO₂C₁₋₃ alkyl and optionally substituted with one to three substituents independently selected from the group consisting of cyano, halogen, C₁₋₄ alkyl, C₁₋₄ alkoxy, C₁₋₄ alkylthio, C₁₋₄ alkylsulfonyl,and trifluoromethyl;
each R² is independently selected from the group consisting of:

hydrogen,

halogen,

hydroxy,

cyano,

amino,

nitro,

C₁₋₄ alkyl, optionally substituted with one to five fluorines,

C₁₋₄ alkoxy, optionally substituted with one to five fluorines,

C₁₋₄ alkylthio, optionally substituted with one to five fluorines,

C₁₋₄ alkylsulfonyl,

carboxy,

C₁₋₄ alkyloxycarbonyl, and

C₁₋₄ alkylcarbonyl;
each R³ is independently selected from the group consisting of:

C₁₋₆ alkyl,

C₂₋₆ alkenyl,

(CH₂)ₙ-phenyl,

(CH₂)ₙ-naphthyl,

(CH₂)ₙ-heteroaryl,

(CH₂)ₙ-heterocyclyl,

(CH₂)ₙC₃₋₇ cycloalkyl,

halogen,

nitro,

(CH₂)ₙOR⁴,

(CH₂)ₙN(R⁴)₂,

(CH₂)ₙC≡N,

(CH₂)ₙCO₂R⁴,

(CH₂)ₙNR⁴SO₂R⁴

(CH₂)ₙSO₂N(R⁴)₂,

(CH₂)ₙS(O)₀₋₂R⁴,

(CH₂)ₙNR⁴C(O)N(R⁴)₂,

(CH₂)ₙC(O)N(R⁴)₂,

(CH₂)ₙNR⁴C(O)R⁴,

(CH₂)ₙNR⁴CO₂R⁴,
(CH₂)ₙC(O)R⁴,
O(CH₂)ₙC(O)N(R⁴)₂,
(CH₂)ₛ-Z-(CH₂)ₜ-phenyl,
(CH₂)ₛ-Z-(CH₂)ₜ-naphthyl,
(CH₂)ₛ-Z-(CH₂)ₜ-heteroaryl,
(CH₂)ₛ-Z-(CH₂)ₜ-heterocyclyl,
(CH₂)ₛ-Z-(CH₂)ₜ—C₃₋₇ cycloalkyl,
(CH₂)ₛ-Z-(CH₂)ₜ—OR⁴,
(CH₂)ₛ-Z-(CH₂)ₜ—N(R⁴)₂,
(CH₂)ₛ-Z-(CH₂)ₜ—NR⁴SO₂R⁴,
(CH₂)ₛ-Z-(CH₂)ₜ—C≡N,
(CH₂)ₛ-Z-(CH₂)ₜ—CO₂R⁴,
(CH₂)ₛ-Z-(CH₂)ₜ—SO₂N(R⁴)₂,
(CH₂)ₛ-Z-(CH₂)ₜ—S(O)₀₋₂R⁴,
(CH₂)ₛ-Z-(CH₂)ₜ—NR⁴C(O)N(R⁴)₂,
(CH₂)ₛ-Z-(CH₂)ₜ—C(O)N(R⁴)₂,
(CH₂)ₛ-Z-(CH₂)ₜ—NR⁴C(O)R⁴,
(CH₂)ₛ-Z-(CH₂)ₜ—NR⁴CO₂R⁴,
(CH₂)ₛ-Z-(CH₂)ₜ—C(O)R⁴,
CF₃,
CH₂CF₃,
OCF₃, and
OCH₂CF₃;
   in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
each R⁴ is independently selected from the group consisting of

hydrogen,

C₁₋₆ alkyl,

(CH₂)ₙ-phenyl,

(CH₂)ₙ-heteroaryl,

(CH₂)ₙ-naphthyl, and

(CH₂)ₙC₃₋₇ cycloalkyl;

wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy; and
each R¹⁴ is hydrogen or C₁₋₃ alkyl.

In one embodiment of the compounds of the present invention, m is 0 or 1. In a class of this embodiment, m is 0.

In a second embodiment of the compounds of the present invention, q and r are both 1, affording a 6-membered piperidine ring.

In a third embodiment of the compounds of the present invention, q is 1 and r is 0, affording a 5-membered pyrrolidine ring.

In a fourth embodiment of the compounds of the present invention, q and r are both 0, affording a 4-membered azetidine ring.

In a fifth embodiment of the compounds of the present invention, X—Y is N—C(O). In a class of this embodiment, Ar is phenyl substituted with one to three R³ substituents as defined above.

In a sixth embodiment of the compounds of the present invention, X—Y is CH—O. In a class of this embodiment, Ar is phenyl substituted with one to three R³ substituents as defined above.

In a seventh embodiment of the compounds of the present invention, X—Y is CH—S(O)ₚ. In a class of this embodiment, Ar is phenyl substituted with one to three R³ substituents as defined above.

In an eighth embodiment of the compounds of the present invention, X—Y is N—CR^{a}R^{b}. In a class of this embodiment, Ar is phenyl substituted with one to three R³ substituents as defined above. In yet another class of this embodiment, R^{a} and R^{b} are hydrogen and Ar is phenyl substituted with one to three R³ substituents.

In a ninth embodiment of the compounds of the present invention, X—Y is CR¹³—CR^{a}R^{b}. In a class of this embodiment, Ar is phenyl substituted with one to three R³ substituents as defined above. In yet another class of this embodiment, R^{a}, R^{b}, and R¹³ are hydrogen and Ar is phenyl substituted with one to three R³ substituents.

In a further embodiment of the compounds of the present invention, R⁵, R⁶, R⁷, R⁸, R⁹, R¹⁰, R¹¹, and R¹² are each hydrogen.

In yet a further embodiment, W is heteroaryl selected from the group consisting of:

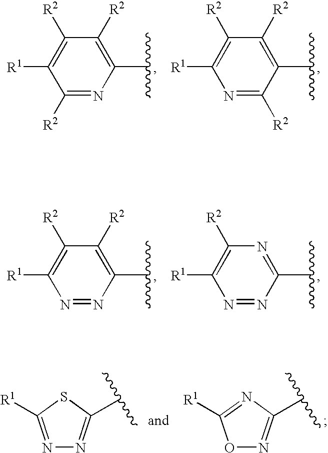

wherein R¹ and R² are as defined above. In a class of this embodiment, each R² is hydrogen.

In a yet a further embodiment, R¹ is pyridin-3-yl or pyrimidin-2-yl, wherein R¹ is monosubstituted with a substituent selected from the group consisting of:

—CO₂H,

—CH₂CO₂H,

—CO₂C₁₋₃ alkyl, and

—CH₂CO₂C₁₋₃ alkyl;

and optionally substituted with one to two substituents independently selected from the group consisting of cyano, halogen, C₁₋₄ alkyl, C₁₋₄ alkoxy, C₁₋₄ alkylthio, C₁₋₄ alkylsulfonyl, and trifluoromethyl.
In a class of this embodiment, R¹ is selected from the group consisting of:

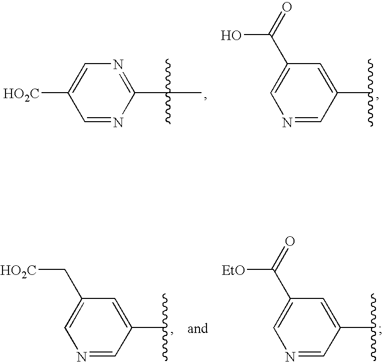

wherein R¹ is optionally substituted with one to two substituents independently selected from the group consisting of halogen, C₁₋₄ alkyl, and trifluoromethyl.

In yet a further embodiment of the compounds of the present invention, q and r are both 0; X—Y is CH—O; W is heteroaryl selected from the group consisting of:

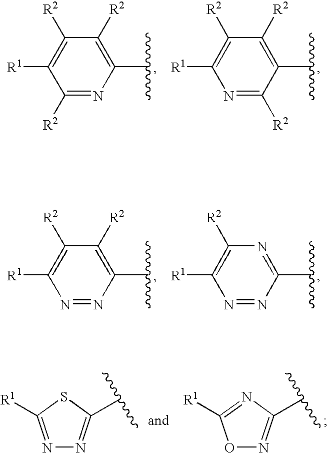

and R¹ is pyridin-3-yl or pyrimidin-2-yl, wherein R¹ is monosubstituted with a substituent selected from the group consisting of:

—CO₂H,

—CH₂CO₂H,

—CO₂C₁₋₃ alkyl, and

—CH₂CO₂C₁₋₃ alkyl;

and optionally substituted with one to two substituents independently selected from the group consisting of cyano, halogen, C₁₋₄ alkyl, C₁₋₄ alkoxy, C₁₋₄ alkylthio, C₁₋₄ alkylsulfonyl, and trifluoromethyl.

In a class of this embodiment, R², R⁵, R⁶, R⁷, R⁸, R⁹, R¹⁰, R¹¹, and R¹² are each hydrogen.

Illustrative, but nonlimiting examples, of compounds of the present invention that are useful as inhibitors of SCD are the following:

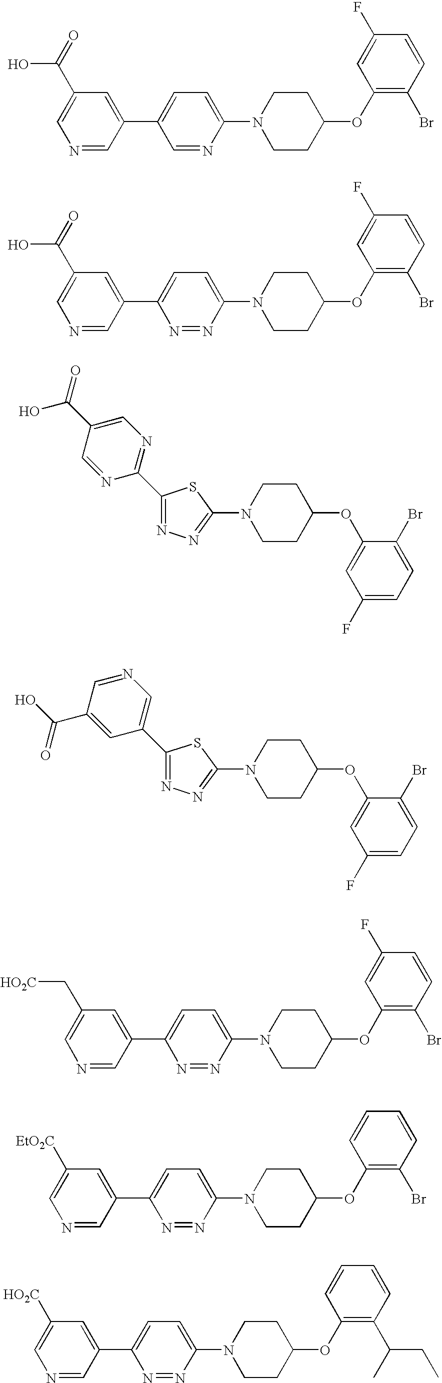

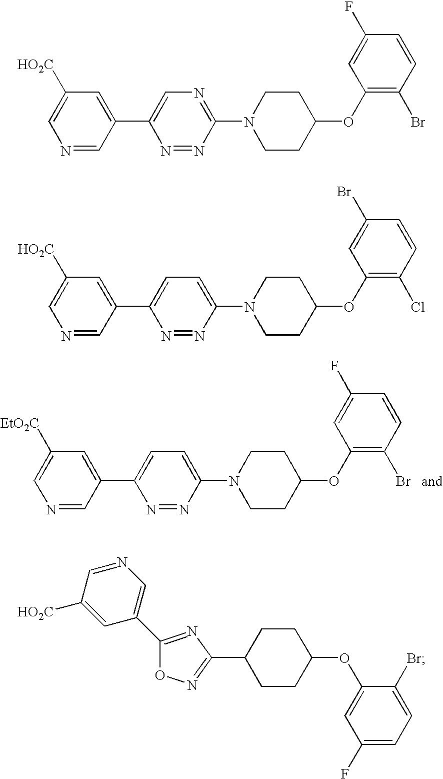

and pharmaceutically acceptable salts thereof.

As used herein the following definitions are applicable.

“Alkyl”, as well as other groups having the prefix “alk”, such as alkoxy and alkanoyl, means carbon chains which may be linear or branched, and combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like. Where the specified number of carbon atoms permits, e.g., from C₃₋₁₀, the term alkyl also includes cycloalkyl groups, and combinations of linear or branched alkyl chains combined with cycloalkyl structures. When no number of carbon atoms is specified, C₁₋₆ is intended.

“Cycloalkyl” is a subset of alkyl and means a saturated carbocyclic ring having a specified number of carbon atoms. Examples of cycloalkyl include cyclopropyl, cyclobutyl, cyclopentyl, cyclohexyl, cycloheptyl, cyclooctyl, and the like. A cycloalkyl group generally is monocyclic unless stated otherwise. Cycloalkyl groups are saturated unless otherwise defined.

The term “alkenyl” shall mean straight or branched-chain alkenes having the specified number of carbon atoms. Examples of alkenyl include vinyl, 1-propenyl, 1-butenyl, 2-butenyl, and the like.

The term “alkoxy” refers to straight or branched chain alkoxides of the number of carbon atoms specified (e.g., C₁₋₆ alkoxy), or any number within this range [i.e., methoxy (MeO—), ethoxy, isopropoxy, etc.].

The term “alkylthio” refers to straight or branched chain alkylsulfides of the number of carbon atoms specified (e.g., C₁₋₆ alkylthio), or any number within this range [i.e., methylthio (MeS—), ethylthio, isopropylthio, etc.].

The term “alkylamino” refers to straight or branched alkylamines of the number of carbon atoms specified (e.g., C₁₋₆ alkylamino), or any number within this range [i.e., methylamino, ethylamino, isopropylamino, t-butylamino, etc.].

The term “alkylsulfonyl” refers to straight or branched chain alkylsulfones of the number of carbon atoms specified (e.g., C₁₋₆ alkylsulfonyl), or any number within this range [i.e., methylsulfonyl (MeSO₂—), ethylsulfonyl, isopropylsulfonyl, etc.].

The term “alkylsulfinyl” refers to straight or branched chain alkylsulfoxides of the number of carbon atoms specified (e.g., C₁₋₆ alkylsulfinyl), or any number within this range [i.e., methylsulfinyl (MeSO—), ethylsulfinyl, isopropylsulfinyl, etc.].

The term “alkyloxycarbonyl” refers to straight or branched chain esters of a carboxylic acid derivative of the present invention of the number of carbon atoms specified (e.g., C₁₋₆ alkyloxycarbonyl), or any number within this range [i.e., methyloxycarbonyl (MeOCO—), ethyloxycarbonyl, or butyloxycarbonyl].

“Aryl” means a mono- or polycyclic aromatic ring system containing carbon ring atoms. The preferred aryls are monocyclic or bicyclic 6-10 membered aromatic ring systems. Phenyl and naphthyl are preferred aryls. The most preferred aryl is phenyl.

“Heterocyclyl” refer to saturated or unsaturated non-aromatic rings or ring systems containing at least one heteroatom selected from O, S and N, further including the oxidized forms of sulfur, namely SO and SO₂. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, 2-oxopiperidin-1-yl, 2-oxopyrrolidin-1-yl, and 2-oxoazetidin-1-yl, and the like.

“Heteroaryl” means an aromatic or partially aromatic heterocycle that contains at least one ring heteroatom selected from O, S and N. Heteroaryls thus includes heteroaryls fused to other kinds of rings, such as aryls, cycloalkyls and heterocycles that are not aromatic. Examples of heteroaryl groups include: pyrrolyl, isoxazolyl, isothiazolyl, pyrazolyl, pyridyl, oxazolyl, oxadiazolyl (in particular, 1,3,4-oxadiazol-2-yl and 1,2,4-oxadiazol-3-yl), thiadiazolyl, thiazolyl, imidazolyl, triazolyl, tetrazolyl, furyl, triazinyl, thienyl, pyrimidyl, benzisoxazolyl, benzoxazolyl, benzothiazolyl, benzothiadiazolyl, dihydrobenzofuranyl, indolinyl, pyridazinyl, indazolyl, isoindolyl, dihydrobenzothienyl, indolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, benzofuranyl, benzothienyl, quinolyl, indolyl, isoquinolyl, dibenzofuranyl, and the like. For heterocyclyl and heteroaryl groups, rings and ring systems containing from 3-15 atoms are included, forming 1-3 rings.

“Halogen” refers to fluorine, chlorine, bromine and iodine. Chlorine and fluorine are generally preferred. Fluorine is most preferred when the halogens are substituted on an alkyl or alkoxy group (e.g. CF₃O and CF₃CH₂O).

Compounds of structural formula I may contain one or more asymmetric centers and can thus occur as racemates and racemic mixtures, single enantiomers, diastereomeric mixtures and individual diastereomers. The present invention is meant to comprehend all such isomeric forms of the compounds of structural formula I.

Compounds of structural formula I may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general structural formula I may be obtained by stereospecific synthesis using optically pure starting materials or reagents of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Some of the compounds described herein contain olefinic double bonds, and unless specified otherwise, are meant to include both E and Z geometric isomers.

Some of the compounds described herein may exist as tautomers, which have different points of attachment of hydrogen accompanied by one or more double bond shifts. For example, a ketone and its enol form are keto-enol tautomers. The individual tautomers as well as mixtures thereof are encompassed with compounds of the present invention.

It will be understood that, as used herein, references to the compounds of structural formula I are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, edetate, edisylate, estolate, esylate, fumarate, gluceptate, gluconate, glutamate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (-COOH) or alcohol group being present in the compounds of the present invention, pharmaceutically acceptable esters of carboxylic acid derivatives, such as methyl, ethyl, or pivaloyloxymethyl, or acyl derivatives of alcohols, such as acetyl, pivaloyl, benzoyl, and aminoacyl, can be employed. Included are those esters and acyl groups known in the art for modifying the solubility or hydrolysis characteristics for use as sustained-release or prodrug formulations.

Solvates, in particular hydrates, of the compounds of structural formula I are included in the present invention as well.

The subject compounds are useful in a method of inhibiting the stearoyl-coenzyme A delta-9 desaturase enzyme (SCD) in a patient such as a mammal in need of such inhibition comprising the administration of an effective amount of the compound. The compounds of the present invention are therefore useful to control, prevent, and/or treat conditions and diseases mediated by high or abnormal SCD enzyme activity.

Thus, one aspect of the present invention concerns a method of treating hyperglycemia, diabetes or insulin resistance in a mammalian patient in need of such treatment, which comprises administering to said patient an effective amount of a compound in accordance with structural formula I or a pharmaceutically salt or solvate thereof.

A second aspect of the present invention concerns a method of treating non-insulin dependent diabetes mellitus (Type 2 diabetes) in a mammalian patient in need of such treatment comprising administering to the patient an antidiabetic effective amount of a compound in accordance with structural formula I.

A third aspect of the present invention concerns a method of treating obesity in a mammalian patient in need of such treatment comprising administering to said patient a compound in accordance with structural formula I in an amount that is effective to treat obesity.

A fourth aspect of the invention concerns a method of treating metabolic syndrome and its sequelae in a mammalian patient in need of such treatment comprising administering to said patient a compound in accordance with structural formula I in an amount that is effective to treat metabolic syndrome and its sequelae. The sequelae of the metabolic syndrome include hypertension, elevated blood glucose levels, high triglycerides, and low levels of HDL cholesterol.

A fifth aspect of the invention concerns a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a compound in accordance with structural formula I in an amount that is effective to treat said lipid disorder.

A sixth aspect of the invention concerns a method of treating atherosclerosis in a mammalian patient in need of such treatment comprising administering to said patient a compound in accordance with structural formula I in an amount effective to treat atherosclerosis.

A seventh aspect of the invention concerns a method of treating cancer in a mammalian patient in need of such treatment comprising administering to said patient a compound in accordance with structural formula I in an amount effective to treat cancer.

A further aspect of the invention concerns a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to treat said condition.

Yet a further aspect of the invention concerns a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, and other conditions and disorders where insulin resistance is a component, in a mammalian patient in need of such treatment comprising administering to the patient a compound in accordance with structural formula I in an amount that is effective to delay the onset of said condition.

Yet a further aspect of the invention concerns a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) metabolic syndrome, and (24) other conditions and disorders where insulin resistance is a component, in a mammalian patient in need of such treatment comprising administering to the patient a compound in accordance with structural formula I in an amount that is effective to reduce the risk of developing said condition.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent, such as a mouse, species can be treated. However, the method can also be practiced in other species, such as avian species (e.g., chickens).

The present invention is further directed to a method for the manufacture of a medicament for inhibiting stearoyl-coenzyme A delta-9 desaturase enzyme activity in humans and animals comprising combining a compound of the present invention with a pharmaceutically acceptable carrier or diluent. More particularly, the present invention is directed to the use of a compound of structural formula I in the manufacture of a medicament for use in treating a condition selected from the group consisting of hyperglycemia, Type 2 diabetes, insulin resistance, obesity, and a lipid disorder in a mammal, wherein the lipid disorder is selected from the group consisting of dyslipidemia, hyperlipidemia, hypertriglyceridemia, hypercholesterolemia, low HDL, and high LDL.

The subject treated in the present methods is generally a mammal, preferably a human being, male or female, in whom inhibition of stearoyl-coenzyme A delta-9 desaturase enzyme activity is desired. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of stearoyl-coenzyme A delta-9 desaturase (SCD) enzyme activity may be demonstrated by the following microsomal and whole-cell based assays:

### I. SCD-Induced Rat Liver Microsome Assay:

The activity of compounds of formula I against the SCD enzyme is determined by following the conversion of radiolabeled-stearoyl-CoA to oleoyl-CoA using SCD1-induced rat liver microsome and a previously published procedure with some modifications (Joshi, et al., J. Lipid Res., 18: 32-36 (1977)). After feeding wistar rats with a high carbohydrate/fat-free rodent diet (LabDiet #5803, Purina) for 3 days, the SCD-induced livers were homogenized (1:10 w/v) in 250 mM sucrose, 1 mM EDTA, 5 mM DTT and 50 mM Tris-HCl (pH 7.5). After a 20 min centrifugation (18,000×g/4° C.) to remove tissue and cell debris, the microsome was prepared by a 100,000×g centrifugation (60 min) with the resulting pellet suspended in 100 mM sodium phosphate, 20% glycerol and 2 mM DTT. Test compound in 2 μL DMSO was incubated for 15 min.at room temperature with 180 μL of the microsome (typically at about 100 μg/mL, in Tris-HCl buffer (100 mM, pH 7.5), ATP (5 mM), Coenzyme A (0.1 mM), Triton X-100 (0.5 mM) and NADH (2 mM)). The reaction was initiated by the addition of 20 μL of [³H]-Stearoyl-CoA (final concentration at 2 μM with the radioactivity concentration at 1 μCi/mL), and terminated by the addition of 150 μL of 1N sodium hydroxide. After 60 min at room temperature to hydrolyze the oleoyl-CoA and stearoyl-CoA, the solution was acidified by the addition of 150 μL of 15% phosphoric acid (v/v) in ethanol supplemented with 0.5 mg/mL stearic acid and 0.5 mg/mL oleic acid. [³H]-oleic acid and [³H]-stearic acid were then quantified on a HPLC that is equipped with a C-18 reverse phase column and a Packard Flow Scintillation Analyzer. Alternatively, the reaction mixture (80 μL) was mixed with a calcium chloride/charcoal aqueous suspension (100 μL of 15% (w/v) charcoal plus 20 μL of 2 N CaCl₂). The resulting mixture was centrifuged to precipitate the radioactive fatty acid species into a stable pellet. Tritiated water from SCD-catalyzed desaturation of 9,10-[³H]-stearoyl-CoA was quantified by counting 50 μL of the supernant on a scintillation counter.

### II. Whole Cell-Based SCD (Delta-9), Delta-5 and Delta-6 Desaturase Assays:

Human HepG2 cells were grown on 24-well plates in MEM media (Gibco cat#11095-072) supplemented with 10% heat-inactivated fetal bovine serum at 37° C. under 5% CO₂ in a humidified incubator. Test compound dissolved in the media was incubated with the subconfluent cells for 15 min at 37° C. [1-¹⁴C]-stearic acid was added to each well to a final concentration of 0.05 μCi/mL to detect SCD-catalyzed [¹⁴C]-oleic acid formation. 0.05 μCi/mL of [1-¹⁴C]-eicosatrienoic acid or [¹⁴C]-linolenic acid plus 10 μM of 2-amino-N-(3-chlorophenyl)benzamide (a delta-5 desaturase inhibitor) was used to index the delta-5 and delta-6 desaturase activities, respectively. After 4 h incubation at 37° C., the culture media was removed and the labeled cells were washed with PBS (3×1 mL) at room temperature. The labeled cellular lipids were hydrolyzed under nitrogen at 65° C. for 1 h using 400 μL of 2N sodium hydroxide plus 50 μL of L-α-phosphatidylcholine (2 mg/mL in isopropanol, Sigma #P-3556). After acidification with phosphoric acid (60 μL), the radioactive species were extracted with 300 μL of acetonitrile and quantified on a HPLC that was equipped with a C-18 reverse phase column and a Packard Flow Scintillation Analyzer. The levels of [¹⁴C]-oleic acid over [¹⁴C]-stearic acid, [¹⁴C]-arachidonic acid over [¹⁴C]-eicosatrienoic acid, and [¹⁴C]-eicosatetraenoic acid (8,11,14,17) over [¹⁴C]-linolenic acid were used as the corresponding activity indices of SCD, delta-5 and delta-6 desaturase, respectively.

The SCD inhibitors of formula I, particularly the compounds of Examples 1 to 38, exhibit an inhibition constant IC₅₀ of less than 1 μM and more typically less than 0.1 μM. Generally, the IC₅₀ ratio for delta-5 or delta-6 desaturases to SCD for a compound of formula I particularly for Examples 1 to 38, is at least about ten or more, and preferably about hundred or more.

### In Vivo Efficacy of Compounds of the Present Invention:

The in vivo efficacy of compounds of formula I was determined by following the conversion of [1-¹⁴C]-stearic acid to [1-¹⁴C]oleic acid in animals as exemplified below. Mice were dosed with a compound of formula I and one hour later the radioactive tracer, [1-¹⁴C]-stearic acid, was dosed at 20 μCi/kg IV. At 3 h post dosing of the compound, the liver was harvested and then hydrolyzed in 10 N sodium hydroxide for 24 h at 80° C., to obtain the total liver fatty acid pool. After phosphoric acid acidification of the extract, the amount of [1-¹⁴C]-stearic acid and [1-¹⁴C]-oleic acid was quantified on a HPLC that was equipped with a C-18 reverse phase column and a Packard Flow Scintillation Analyzer.

The subject compounds are further useful in a method for the prevention or treatment of the aforementioned diseases, disorders and conditions in combination with other agents.

The compounds of the present invention may be used in combination with one or more other drugs in the treatment, prevention, suppression or amelioration of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also include therapies in which the compound of formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of formula I, and either administered separately or in the same pharmaceutical composition, include, but are not limited to:

(a) dipeptidyl peptidase-IV (DPP-4) inhibitors;

(b) insulin sensitizers including (i) PPARγ agonists, such as the glitazones (e.g. PPARα agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators.

Dipeptidyl peptidase-IV inhibitors that can be combined with compounds of structural formula I include those disclosed in U.S. Pat. No. 6,699,871; WO 02/076450 (3 Oct. 2002); WO 03/004498 (16 Jan. 2003); WO 03/004496 (16 Jan. 2003); EP 1 258 476 (20 Nov. 2002); WO 02/083128 (24 Oct. 2002); WO 02/062764 (15 Aug. 2002); WO 03/000250 (3 Jan. 2003); WO 03/002530 (9 Jan. 2003); WO 03/002531 (9 Jan. 2003); WO 03/002553 (9 Jan. 2003); WO 03/002593 (9 Jan. 2003); WO 03/000180 (3 Jan. 2003); WO 03/082817 (9 Oct. 2003); WO 03/000181 (3 Jan. 2003); WO 04/007468 (22 Jan. 2004); WO 04/032836 (24 Apr. 2004); WO 04/037169 (6 May 2004); and WO 04/043940 (27 May 2004). Specific DPP-IV inhibitor compounds include sitagliptin (MK-0431); vildagliptin (LAF 237); denagliptin; P93/01; saxagliptin (BMS 477118); RO0730699; MP513; SYR-322: ABT-279; PHX1149; GRC-8200; and TS021.

Antiobesity compounds that can be combined with compounds of structural formula I include fenfluramine, dexfenfluramine, phentermine, sibutramine, orlistat, neuropeptide Y₁ or Y₅ antagonists, cannabinoid CB1 receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists. For a review of anti-obesity compounds that can be combined with compounds of structural formula I, see S. Chaki et al., “Recent advances in feeding suppressing agents: potential therapeutic strategy for the treatment of obesity,” Expert Opin. Ther. Patents, 11:1677-1692 (2001); D. Spanswick and K. Lee, “Emerging antiobesity drugs,” Expert Opin. Emerging Drugs, 8: 217-237 (2003); and J. A. Fernandez-Lopez, et al., “Pharmacological Approaches for the Treatment of Obesity,” Drugs, 62: 915-944 (2002).

Neuropeptide Y5 antagonists that can be combined with compounds of structural formula I include those disclosed in U.S. Pat. No. 6,335,345 (1 Jan. 2002) and WO 01/14376 (1 Mar. 2001); and specific compounds identified as GW 59884A; GW 569180A; LY366377; and CGP-71683A.

Cannabinoid CB1 receptor antagonists that can be combined with compounds of formula I include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; PCT Publication WO 03/087037; PCT Publication WO 04/048317; PCT Publication WO 03/007887; PCT Publication WO 03/063781; PCT Publication WO 03/075660; PCT Publication WO 03/077847; PCT Publication WO 03/082190; PCT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and PCT Publication WO 01/64634.

Melanocortin-4 receptor (MC4R) agonists useful in the present invention include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02//092566, WO 03/-093234, WO 03/095474, and WO 03/104761.

One particular aspect of combination therapy concerns a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a compound of structural formula I and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

In another aspect of the invention, a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a compound of structural formula I and an HMG-CoA reductase inhibitor.

In another aspect of the invention, a method for delaying the onset or reducing the risk of developing atherosclerosis in a human patient in need of such treatment is disclosed comprising administering to said patient an effective amount of a compound of structural formula I and an HMG-CoA reductase inhibitor.

More particularly, a method for delaying the onset or reducing the risk of developing atherosclerosis in a human patient in need of such treatment is disclosed, wherein the HMG-CoA reductase inhibitor is a statin selected from the group consisting of: lovastatin, simvastatin, pravastatin, cerivastatin, fluvastatin, atorvastatin, and rosuvastatin.

In another aspect of the invention, a method for delaying the onset or reducing the risk of developing atherosclerosis in a human patient in need of such treatment is disclosed, wherein the HMG-Co A reductase inhibitor is a statin and further comprising administering a cholesterol absorption inhibitor.

More particularly, in another aspect of the invention, a method for delaying the onset or reducing the risk of developing atherosclerosis in a human patient in need of such treatment is disclosed, wherein the HMG-Co A reductase inhibitor is a statin and the cholesterol absorption inhibitor is ezetimibe.

In another aspect of the invention, a pharmaceutical composition is disclosed which comprises:
(1) a compound of structural formula I;
(2) a compound selected from the group consisting of:

(a) dipeptidyl peptidase IV (DPP-IV) inhibitors;

(b) insulin sensitizers including (i) PPARγ agonists, such as the glitazones (e.g. troglitazone, pioglitazone, englitazone, MCC-555, rosiglitazone, balaglitazone, and the like) and other PPAR ligands, including PPARα/γ dual agonists, such as KRP-297, muraglitazar, naveglitazar, Galida, TAK-559, PPARA agonists, such as fenofibric acid derivatives (gemfibrozil, clofibrate, fenofibrate and bezafibrate), and selective PPARγ modulators (SPPARγM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-IB (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) α-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (NN-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPARδ agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, β₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11β-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) acetyl CoA carboxylase-1 and/or -2 inhibitors; and

(u) AMPK activators; and
(3) a pharmaceutically acceptable carrier.

When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracistemal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally- occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.) The pharmaceutical composition and method of the present invention may further comprise other therapeutically active compounds as noted herein which are usually applied in the treatment of the above mentioned pathological conditions.

In the treatment or prevention of conditions which require inhibition of stearoyl-CoA delta-9 desaturase enzyme activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0. I to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 mg of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 4 times per day, preferably once or twice per day.

When treating or preventing diabetes mellitus and/or hyperglycemia or hypertriglyceridemia or other diseases for which compounds of the present invention are indicated, generally satisfactory results are obtained when the compounds of the present invention are administered at a daily dosage of from about 0.1 mg to about 100 mg per kilogram of animal body weight, preferably given as a single daily dose or in divided doses two to six times a day, or in sustained release form. For most large mammals, the total daily dosage is from about 1.0 mg to about 1000 mg, preferably from about 1 mg to about 50 mg. In the case of a 70 kg adult human, the total daily dose will generally be from about 7 mg to about 350 mg. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### List of Abbreviations:

Alk=alkyl
APCI=atmospheric pressure chemical ionization
Ar=aryl
Boc=tert-butoxycarbonyl
br=broad
d=doublet
DBU=1,8-diazabicyclo[5.4.0]undec-7-ene
DMF=N,N-dimethylformamide
DAST=diethylaminosulfur trifluoride
Deoxofluor®=bis(2-methoxyethyl)aminosulfur trifluoride
DEBAL-H=diisobutylaluminum hydride
DMSO=dimethyl sulfoxide
ESI=electrospray ionization
EtOAc=ethyl acetate
m=multiplet
m-CPBA=3-chloroperoxybenzoic acid
MeOH=methyl alcohol
MS=mass spectroscopy
NaHMDS=sodium bis(trimethylsilyl)amide
NMP=1-methyl-2-pyrrolidinone
NMR=nuclear magnetic resonance spectroscopy
PG=protecting group
rt=room temperature
s=singlet
t=triplet
THF=tetrahydrofuran
TLC=thin-layer chromatography
TsOH=toluene-4-sulfonic acid

### Preparation of Compounds of the Invention:

The compounds of structural formula I can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESMS).

### Method A:

A mixture of 2,5-dibromopyridine 1 and 4-hydroxypiperidine 2 is heated to provide intermediate compound 3.

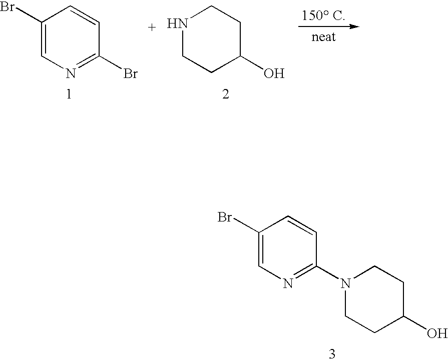

### Method B:

A mixture of the intermediate compound 3 and 2-fluorobenzotrifluoride 4 in DMF is treated with potassium tert-butoxide at elevated temperature to give the bromide 5.

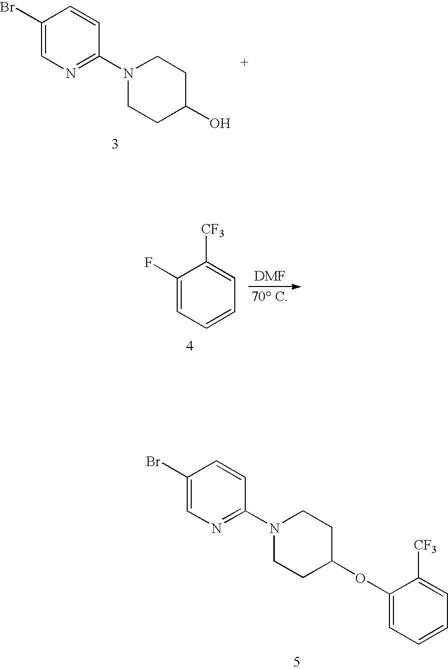

### Method C:

The bromide 5 is treated with bis(pinacolato)diboron, PdCl₂dppf, and KOAc in DMF with heating to afford the boronate 6.

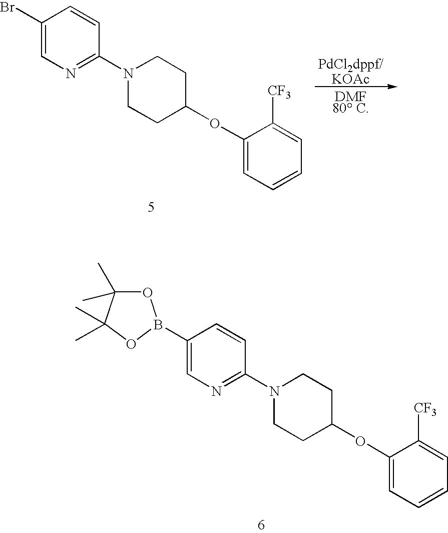

### Method D:

The boronate 6 is reacted with a six-membered heteroaryl halide containing one or two nitrogens, such as ethyl 5-bromonicotinate 7 and a palladium catalyst, such as Pd(OAc)₂ and (Ph₃P)₄Pd, in the presence of base. The ester is then hydrolyzed with NaOH to provide 8. This method can be extended to various halopyridinecarboxylic acid esters, halopyridineacetic acid esters, and halopyridinepropionic acid esters represented by formula 9 to give 10.

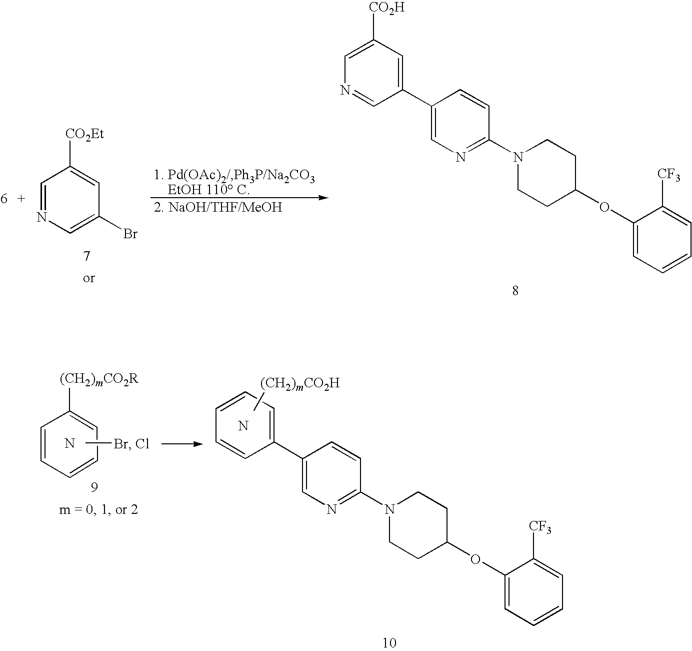

### Method E:

The methods A and B can be applied to other cyclic amines such as 11 to provide alcohols 12 which can be converted to 13.

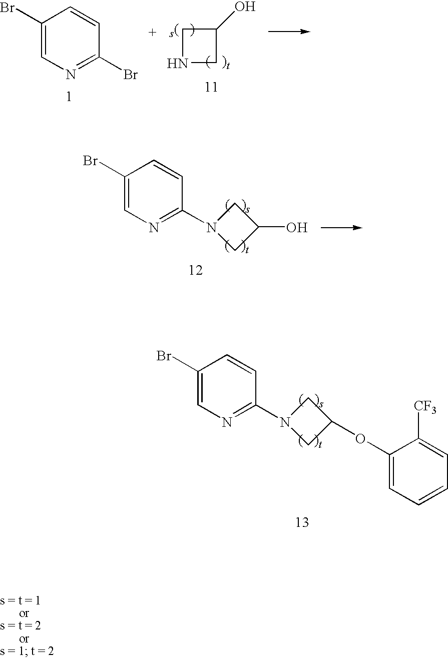

### Method F:

Alcohols 12 can be converted to phenyl ethers 15 via a Mitsunobu reaction with optionally substituted phenols 14.

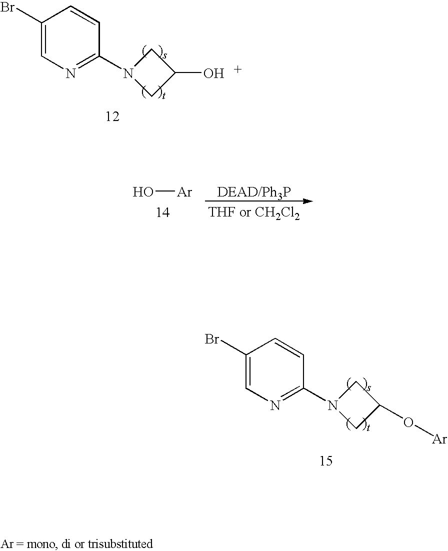

### Method G:

The alcohol 12 can be protected with a silyl group to give 16 which in turn can be converted to boronate 17 using Method C. Palladium-mediated cross-coupling reaction with 17 and an appropriately substituted halopyridine or halopyrimidine can be accomplished using Method D. Removal of the silyl group followed by aryl coupling with either Method B and F provides compounds of the present invention.

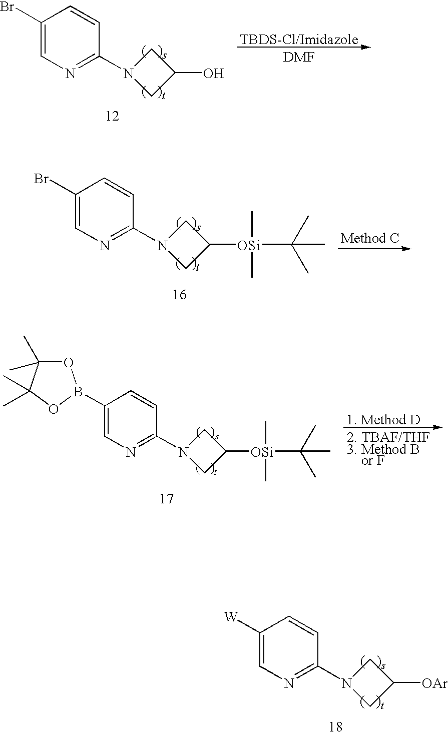

### Method H:

The boronate 6 is reacted with 2-chloropyrimidine-5-carboxylic acid 19 (J. Med. Chem. 2001, 44, 3369-3377) with a catalytic amount of palladium to give 20.

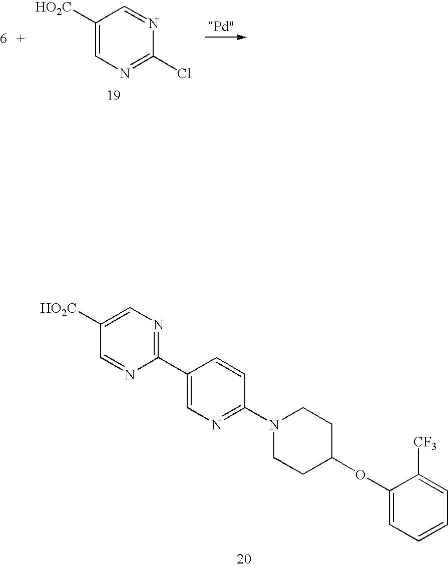

### Method I:

A mixture of 3,6-dichloropyridazine 21 is heated with the cyclic amino alcohol in the presence of a catalytic amount of acid in a polar solvent, such as water and ethanol, to provide compound 22.

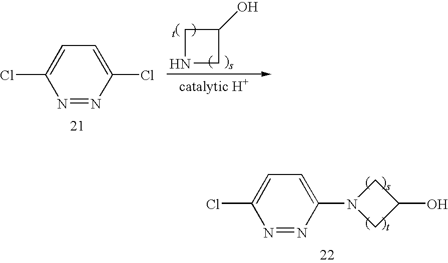

### Method J:

A mixture of the boronic or boronate ester 23 and 3-chloropyridazine 22 is heated in the presence of a palladium catalyst (such as Pd₂dba₃, PdCl₂, Pd(OAc)₂ and [(allyl)PdCl]₂), a phosphine ligand (such as PPh₃, PCy₃, P(t-Bu)₃, and biphenylP(Cy)₂), and a base (such as K₃PO₄, Cs₂CO₃, CsF and KOt-Bu) and a polar solvent to provide the cross-coupled product 24.

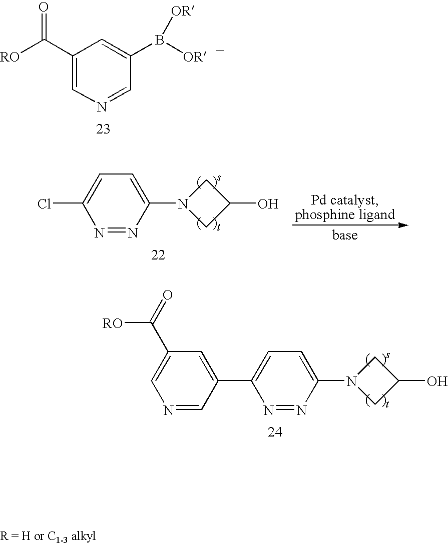

### Method K:

A mixture of pyridazine alcohol 24 and phenol 25 in a solvent, such as THF and dioxane, is reacted with an azodicarboxylate (such as diethyl azodicarboxylate, diisopropyl azodicarboxylate, and 1,1′-(azodicarbonyl)dipiperidine) and a phosphine reagent (such as PPh₃ and Pi-Bu₃) at temperatures ranging from 25° C. to 80° C. Concentration and purification followed by basic hydrolysis of the ester yields the desired compounds 26.

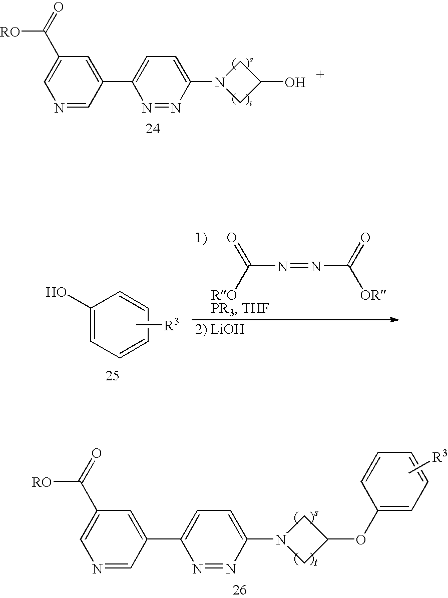

### Method L:

An appropriately substituted amino heteroaryl bromide 27 is reacted with an appropriately substituted cyclic amine 28 in the presence of a base, such as 1,8-diazabicyclo[5.4.0]undec-7-ene (DBU) or an alkali metal (K, Na, Cs) carbonate, in a solvent, such as DMF, THF and EtOH, at a temperature range of about room temperature to about refluxing temperature. Precipitation of the product by the addition of water or extractive work up and purification by flash column chromatography gives the desired amino heteroaryl 29. Reaction of amino heteroaryl 29 with copper (II) bromide and t-butyl nitrite in a solvent such as acetonitrile at a temperature range of about room temperature to about refluxing temperature followed by extractive work up and purification by flash column chromatography gives the desired heteroaryl bromide 30. Suzuki coupling of the heteroaryl bromide 30 with an appropriate carboxy-heteroaryl boronate ester 31 in the presence of palladium (II) and aqueous Na₂CO₃ or K₃PO₄ in a solvent, such as DMF and NMP, at a refluxing temperature followed by extractive work up and purification by flash column chromatography gives the desired heteroaryl ester 32. Hydrolysis of the heteroaryl ester 32 with aqueous NaOH or LiOH in a solvent such as THF and MeOH at a temperature range of about room temperature to about refluxing temperature followed by extractive work up and purification by flash column chromatography or recrystallization affords the heteroaryl carboxylic acid 33.

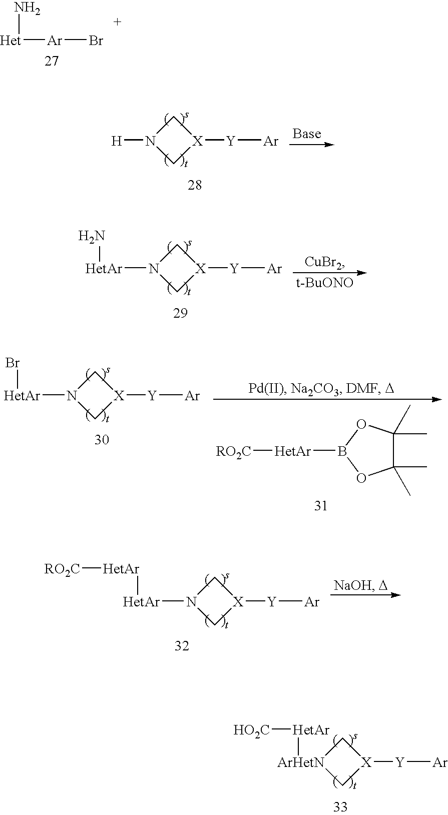

### Method M:

Ethyl 5-bromonicotinate 34 is converted to the aryltin derivative 35 with hexamethylditin in the presence of a palladium catalyst. The tin derivative 35 is then reacted with the chloropyridazine 22 and a palladium catalyst, such as palladium(I) tri-tertbutylphosphine bromide dimer, to provide alcohol 36. Alcohol 36 can be converted to compounds of the present invention utilizing Methods B or F.

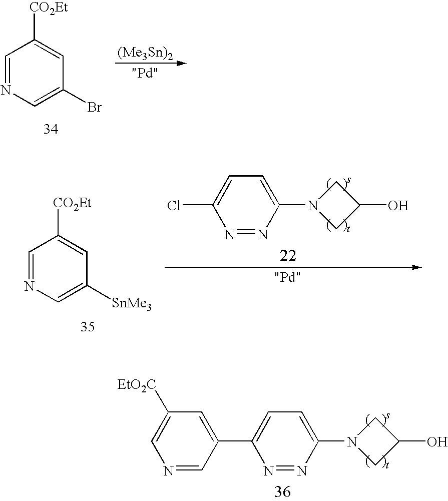

The following Examples are provided to illustrate the invention and are not to be construed as limiting the scope of the invention in any manner.

### Example 1

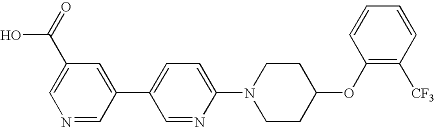

### 6′-(4-{[2-(Trifluoromethyl)phenyl]oxy}-1-piperidinyl)-3,3′-bipyridine-5-carboxylic acid

### Step 1: 1-(5-Bromo-2-pyridinyl)-4-piperidinol

A mixture of 2,5-dibromopyridine and 4-hydroxypiperidine (2.2 equiv.) was heated at 150° C. for 0.5 h. The reaction mixture was then cooled followed by the addition of ethyl acetate and 10% aqueous NaOH until neutral pH. The organic phase was separated and dried over Na₂SO₄ to provide, after evaporation, the title compound as a white solid.

### Step 2: 5-Bromo-2-(4-{[2-(trifluoromethyl)phenyl]}-1-piperidinyl)pyridine

To a solution of 1-(5-bromo-2-pyridinyl)-4-piperidinol in DMF (0.95 M) were added 1 M potassium tert-butoxide (1.15 equiv.) and 2-fluorobenzotrifluoride (1.5 equiv.). After a period of 18 h at 70° C., the reaction mixture was partitioned between ethyl acetate and aqueous NH₄Cl. The organic phase was separated, dried over Na₂SO₄ and evaporated. The title compound was purified over silica gel eluting with 30% ethyl acetate in hexane.

### Step 3: 5-(4,4,5,5-Tetramethyl-1,3,2-dioxaborolan-2-yl)-2-{4-[2-(trifluoromethyl)phenoxy]piperidin-1-yl}pyridine

A mixture of 5-bromo-2-(4-{[2-(trifluoromethyl)phenyl]}-1-piperidinyl)pyridine, bis(pinacolato)diboron (1.5 equiv.), PdCl₂dppf (0.07 equiv.), and KOAc (3.9 equiv.) in DMF (0.13 M) was heated at 80° C. After a period of 18 h, the reaction mixture was partitioned between ether and water. The organic phase was separated, dried over Na₂SO₄ and evaporated. The title compound was purified by flash chromatography eluting with 50% ethyl acetate in hexane.

### Step 4: Ethyl 6′-{4-[2-(trifluoromethyl)phenoxy]piperidin-1-yl}-3,3′-bipyridine-5-carboxylate

A mixture 5-(4,4,5,5-tetramethyl-1,3,2-dioxaborolan-2-yl)-2-{4-[2-(trifluoromethyl)phenoxy]piperidin-1-yl}pyridine, ethyl 5-bromonicotinate (2.5 equiv.), Pd(Ph₃P)₄ (0.1 equiv.), 2M Na₂CO₃ (3.0 equiv.) in DMF (0.08 M) was heated at 100° C. After a period of 4 h, the reaction mixture was partioned between ethyl acetate and water. The organic phase was separated, dried over Na₂SO₄ and evaporated. The title compound was purified by flash chromatography eluting with 40% ethyl acetate to 50% ethyl acetate in hexane.

### Step 5: 6′-(4-{[2-(Trifluoromethyl)phenyl]oxy}-1-piperidinyl)-3,3′-bipyridine-5-carboxylic acid

A solution of ethyl 6′-{4-[2-(trifluoromethyl)phenoxy]piperidin-1-yl}-3,3′-bipyridine-5-carboxylate (0.03 M) in MeOH:THF:NaOH 1M (1:1:1) was stirred for 2 h at room temperature. The title compound was purified by reverse phase HPLC using a C₁₈ CombiPrep ODS-AM column (gradient: 60% H₂O in CH₃CN to 5% H₂O in CH₃CN over 8 min). MS: m/z 443.9 (ESI+).

### Example 2

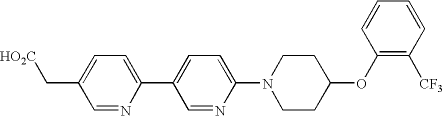

### [6′-(4-{[2-(Trifluoromethyl)phenyl]oxy}-1-piperidinyl)-2,3′-bipyridin-5-yl]acetic acid

### Step 1: [6′-(4-{[2-(Trifluoromethyl)phenyl]oxy}-1-piperidinyl)-2,3′-bipyridin-5yl]acetic acid

Ethyl(6-chloro-pyridin-3-yl)-acetate (1.5 eq) was treated with a mixture of 5-(4,4,5,5-tetramethyl-1,3,2-dioxaborolan-2-yl)-2-{4-[2-(trifluoromethyl)phenoxy]piperidin-1-yl}pyridine (from step 3 of example 1), Pd(OAc)₂ (0.017 equiv.), Ph₃P (0.05 equiv.), and 2M Na₂CO₃ (4.5 equiv.), and the mixture was heated at 80° C. After a period of 4 h, the reaction mixture was hydrolyzed with 2M aqueous LiOH (5 eq) for 3 h at 22° C. The solution was neutralized with the addition of formic acid (30 eq) and concentrated. The residue was suspended in DMSO (0.04 M) and centrifuged. The supernatant was purified by reverse phase HPLC using a C₁₈ CombiPrep ODS-AM column (gradient: 60% H₂O in CH₃CN to 5% H₂O in CH₃CN over 8 min) to obtain the title compound. MS: m/z 458.5 (ESI+).

### Example 3

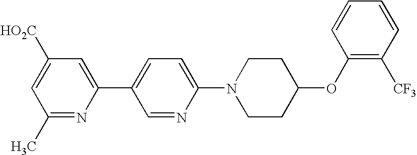

### 6-Methyl-6′-(4-{[2-(trifluoromethyl)phenyl]oxy}-1-piperidinyl)-2,3′-bipyridine-4-carboxylic acid

### Step 1: Methyl 2-chloro-6-methyl isonicotinate

2-Chloro-6-methyl isonicotinic acid was treated in ethanol (0.04 M) with diazomethane in ether to obtain the methyl ester derivative. The solution was concentrated to afford the title compound.

### Step 2: 6-Methyl-6′-(4-{[2-(trifluoromethyl)phenyl]oxy}-1-piperidinyl)-2,3′-bipyridine-4-carboxylic acid

The title compound was prepared as described for example 2, step 1, replacing the ethyl(6-chloro-pyridin-3-yl)-acetate by methyl 2-chloro-6-methyl-isonicotinate in ethanol solution obtained in step 1. MS: m/z 458.5 (ESI+).

### Example 4

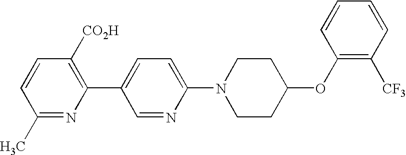

### 6-Methyl-6′-(4-{[2-(trifluoromethyl)phenyl]oxy}-1-piperidinyl)-2,3′-bipyridine-3-carboxylic acid

### Step 1: 6-Methyl-6′-(4-{[2-(trifluoromethyl)phenyl]oxy}-1-piperidinyl)-2,3′-bipyridine-3-carboxylic acid

The title compound was prepared as described for example 2, step 1, replacing the ethyl(6-chloro-pyridin-3-yl)-acetate by methyl 2-chloro-6-methyl-nicotinate that was prepared from 2-chloro-6-methyl-nicotinic acid as described in example 3, step 1. MS: m/z 458.5 (ESI+).

### Example 5

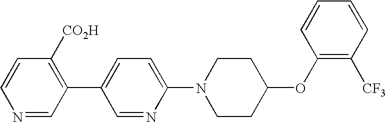

### 6′-(4-{[2-(Trifluoromethyl)phenyl]oxy}-1-piperidinyl)-2,3′-bipyridine-3-carboxylic acid

### Step 1: 6′-(4-{[2-(Trifluoromethyl)phenyl]oxy}-1-piperidinyl)-2,3′-bipyridine-3-carboxylic acid

The title compound was prepared as described in example 2, step 1, replacing the ethyl(6-chloro-pyridin-3-yl)-acetate by methyl 3-bromo-isonicotinate that was prepared from 3-bromo-isonicotinic acid as described in example 3, step 1. MS: m/z 444.4 (ESI+).

### Example 6

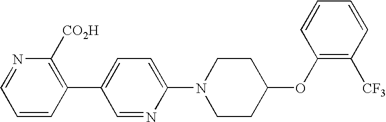

### 6′-(4-{[2-(Trifluoromethyl)phenyl]oxy}-1-piperidinyl)-3,3′-bipyridine-2-carboxylic acid

### Step 1: 6′-(4-{[2-(Trifluoromethyl)phenyl]oxy}-1-piperidinyl)-3,3′-bipyridine-2-carboxylic acid

The title compound was prepared as described in example 2, step 1, replacing the ethyl(6-chloro-pyridin-3-yl)-acetate by methyl 3-bromo-picolinate that was prepared from 3-bromo-picolinic acid as described in example 3, step 1. MS: m/z 444.4 (ESI+).

### Example 7

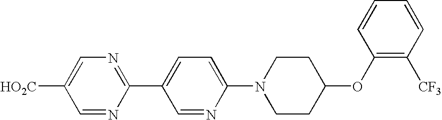

### 2-(6-(4-{[2-(Trifluoromethyl)phenyl]oxy})pyridin-3-yl)pyrimidine-5-carboxylic acid

The title compound was prepared as described in example 2, step 1, replacing the ethyl(6-chloro-pyridin-3-yl)-acetate by methyl 2-chloro-pyrimidine-5-carboxylate that was prepared from 2-chloro-pyrimidine-5-carboxylic acid as described in example 3, step 1. MS: m/z 445.4 (ESI+). Alternatively, the title compound was prepared from 2-chloropyrimidine-5-carboxylic acid, 5-(4,4,5,5-tetramethyl-1,3,2-dioxaborolan-2-yl)-2-{4-[2-(trifluoromethyl)phenoxy]piperidin-1-yl}pyridine (1.4 equiv.), NaHCO₃ (1.5 equiv.), (Ph₃P)₄Pd (0.1 equiv) in DMF/H₂O (1/1) (0.1M).

### Example 8

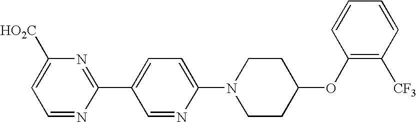

### 2-(6-(4-{[2-(Trifluoromethyl)phenyl]oxy})pyridin-3-yl)pyrimidine-4-carboxylic acid

The title compound was prepared as described in example 2, step 1, replacing the ethyl(6-chloro-pyridin-3-yl)-acetate by methyl 2-chloro-pyrimidine-4-carboxylate that was prepared from 2-chloro-pyrimidine-4-carboxylic acid as described in example 3, step 1. MS: m/z 445.4 (ESI+).

### Example 9

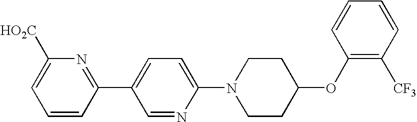

### 4-{[2-(Trifluoromethyl)phenyl]oxy}-1-piperidinyl)-2,3′-bipyridine-6-carboxylic acid

The title compound was prepared as described in example 2, step 1, replacing the ethyl(6-chloro-pyridin-3-yl)-acetate by methyl 6-bromo-picolinate that was prepared from 6-bromo-picolinic acid as described in example 3, step 1. MS: m/z 444.4 (ESI+).

### Example 10

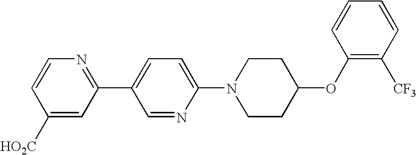

### 6′-(4-{[2-(Trifluoromethyl)phenyl]oxy}-1-piperidinyl)-2,3′-bipyridine-4-carboxylic acid

The title compound was prepared as described in example 2, step 1, replacing the ethyl(6-chloro-pyridin-3-yl)-acetate by methyl 2-bromo-isonicotinate that was prepared from 2-bromo-isonicotinic acid as described in example 3, step 1. MS: m/z 444.4 (ESI+).

### Example 11

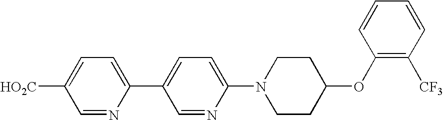

### 6′-(4-{[2-(Trifluoromethyl)phenyl]oxy}-1-piperidinyl)-2,3′-bipyridine-5-carboxylic acid

The title compound was prepared as described in example 2, step 1, replacing the ethyl(6-chloro-pyridin-3-yl)-acetate by methyl 2-chloro-nicotinate. MS: m/z 444.4 (ESI+).

### Example 12

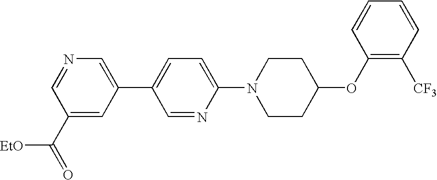

### Ethyl 6′-{4-[2-(trifluoromethyl)phenoxy]piperidin-1-yl}-3,3′-bipyridine-5-carboxylate

The title compound was prepared as described in example 1, step 4. MS: m/z 472.2 (ESI+).

### Example 13

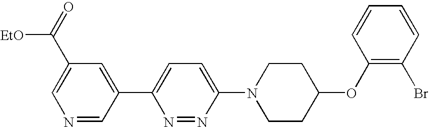

### Ethyl 5-{6-[4-(2-bromophenoxy)piperidin-1-yl]pyridazin-3-yl}nicotinate

### Step 1: 1-(6-Chloropyridazin-3-yl)piperidin-4-ol

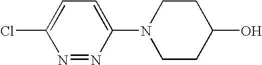

Into a round-bottom flask equipped with a magnetic stirbar and a reflux condenser was added 3,6-dichloropyridazine (1 equiv.), 4-hydroxypiperidine (1.3 equiv.) and water (200 mL). The suspension was treated with dropwise addition of concentrated hydrochloric acid (1.02 mL, 0.1 equiv.) and the suspension heated to 80° C. for 24 h. The resulting orange solution was cooled to room temperature and basified to pH=11 with 10 M aqueous sodium hydroxide. The resulting suspension was poured into a separatory funnel containing 1 M aqueous NaOH and extracted three times with ethyl acetate. The combined organic layers were washed with brine, dried over MgSO₄, filtered and concentrated under reduced pressure. The resulting yellow solid was triturated in ethyl acetate/diethyl ether and filtered through Whatman #1 filter paper on a Hirsch funnel to give the title compound as a yellow solid.

### Step 2: Ethyl 5-[6-(4-hydroxypiperidin-1-yl)pyridazin-3-yl]nicotinate

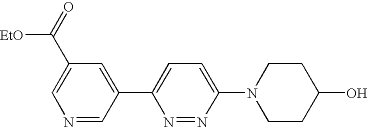

Into a flame-dried Schlenk flask equipped with a magnetic stirbar and under a N₂ atmosphere was added 3-(ethoxycarbonyl)pyridine-5-boronic acid pinacol ester (1 equiv.), 1-(6-chloropyridazin-3-yl)piperidin-4-ol (1.1 equiv), Pd₂(dba)₃ (0.01 equiv.) and tricyclohexylphosphine (0.025 equiv.). The flask was evacuated and back-filled with N₂ (repeated 3 times). The solids were suspended in dioxane (0.5 M) and then an aqueous solution of tribasic potassium phosphate (1.7 equiv.) was added. The mixture was heated to 100° C. in an oil bath for 3 h. The mixture was cooled, poured into a separatory funnel containing pH 5 buffer and the mixture was extracted three times with ethyl acetate. The combined organic layers were washed with brine, dried over MgSO₄, filtered and the solvent was evaporated under reduced pressure. Purification by column chromatography on silica gel (eluting with 5% MeOH in ethyl acetate) gave the indicated product as a beige solid.

### Step 3: Ethyl 5-{6-[4-(2-bromophenoxy)piperidin-1-yl]pyridazin-3-yl}nicotinate

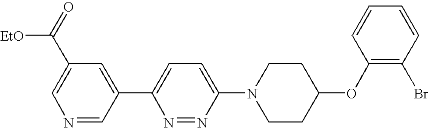

Into a round-bottom flask equipped with a magnetic stirbar was added ethyl 5-[6-(4-hydroxypiperidin-1-yl)pyridazin-3-yl]nicotinate (1 equiv.), 2-bromophenol (1.3 equiv.), diethyl azodicarboxylate (1.3 equiv.) and triphenylphosphine (1.3 equiv.) in THF (1 M). The thick suspension was sonicated for 20 min, becoming a thick orange solution. The reaction mixture was purified by column chromatography on silica gel (eluting with 50% ethyl acetate in hexanes) to afford the desired product as a white solid.

¹H NMR (400 MHz, CDCl₃) δ 9.41 (s, 1H); 9.26 (s, 1H); 8.95 (s, 1H), 7.76-7.65 (m, 2H), 7.62-7.54 (m, 1H), 7.09 (d, J=9.5 Hz, 1H), 7.00 (d, J=8.5 Hz, 1H), 6.90 (t, J=7.5 Hz, 1H), 4.80-4.70 (m, 1H), 4.47 (q, J=7.14 Hz, 2H), 4.02-3.96 (m, 4H), 2.10-2.05 (m, 4H), 1.46 (t, J=7.0 Hz, 3H).

MS (ESI, Q⁺): m/z 483, 485 (M+1 for ⁷⁹Br and ⁸¹Br).

### Example 14

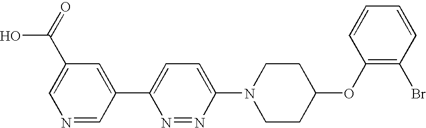

### 5-{6-[4-(2-Bromophenoxy)piperidin-1-yl]pyridazin-3-yl}nicotinic acid

Into a round-bottom flask equipped with a magnetic stirbar and reflux condenser was added ethyl 5-{6-[4-(2-bromophenoxy)piperidin-1-yl]pyridazin-3-yl}nicotinate (1 equiv.), methanol (0.04 M) and 1 M aqueous sodium hydroxide (7.5 equiv.). The resulting suspension was heated to 100° C. for 2 h, and then cooled to room temperature. The reaction mixture was concentrated and poured into a separatory funnel containing pH 5 buffer and extracted three times with ethyl acetate. The combined organic layers were washed with brine, dried over MgSO₄, filtered and concentrated under reduced pressure to yield the desired product as a white solid.

¹H NMR (400 MHz, DMSO-d6): δ 9.42 (s, 1H), 9.09 (s, 1H), 8.85 (s, 1H), 8.15 (d, J=9.5 Hz, 1H), 7.59 (d, J=1.5 Hz, 1H), 7.47 (d, J=9.5 Hz, 1H), 7.39-7.33 (m, 1H), 7.30-7.24 (m, 1H), 6.92 (t, J=7.5 Hz, 1H), 4.85 (bs, 1H), 4.02-3.94 (m, 2H), 3.81-3.73 (m, 2H), 2.06-1.98 (m, 2H), 1.80-1.74 (m, 2H). MS (ESI, Q⁺) m/z 454, 456 (M+1 for ⁷⁹Br and ⁸¹Br).

### Example 15

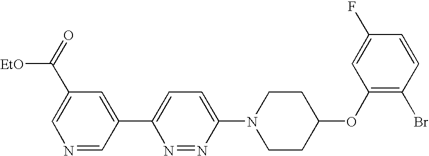

### Ethyl 5-{6-[4-(2-bromo-5-fluorophenoxy)piperidin-1-yl]pyridazin-3-yl}nicotinate

Into a round-bottom flask equipped with a magnetic stirbar was added ethyl 5-[6-(4-hydroxypiperidin-1-yl)pyridazin-3-yl]nicotinate (1 equiv.), 2-bromo-5-fluorophenol (1.3 equiv.), diethyl azodicarboxylate (1.3 equiv.) and triphenylphosphine (1.3 equiv.) in THF (1 M). The thick suspension was sonicated for 20 min, becoming a thick orange solution. The reaction mixture was purified by column chromatography on silica gel (eluting with 40% ethyl acetate in hexanes) to afford the desired product as a white solid.

¹H NMR (400 MHz, CDCl₃) δ 9.40 (s, 1H), 9.26 (s, 1H), 8.94 (s, 1H), 7.77-7.72 (m, 1H), 7.55-7.49 (m, 1H), 7.09 (d, J=9.5 Hz, 1H), 6.72 (dd, J=10.5, 3.0 Hz, 1H), 6.64 (ddd, J=9.0, 8.0, 3.0 Hz, 1H), 4.74-4.68 (m, 1H), 4.46 (q, J=7.0 Hz, 2H); 4.03-3.91 (m, 4H), 2.12-2.04 (m, 4H), 1.45 (t, J=7.0 Hz, 3H). MS (ESI, Q+) m/z 501, 503 (M+1 for ⁷⁹Br and ⁸¹Br).

### Example 16

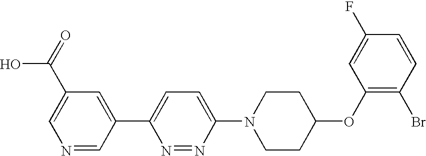

### 5-{6-[4-(2-Bromo-5-fluorophenoxy)piperidin-1-yl]pyridazin-3-yl}nicotinic acid

Into a round-bottom flask equipped with a magnetic stirbar and reflux condenser was added ethyl 5-{6-[4-(2-bromo-5-fluorophenoxy)piperidin-1-yl]pyridazin-3-yl}nicotinate (1 equiv.), methanol (0.04 M) and 1 M aqueous sodium hydroxide (5 equiv.). The resulting suspension was heated to 100° C. for 2 h, and then cooled to room temperature. The reaction mixture was concentrated and poured into a separatory funnel containing pH 5 buffer and extracted with ethyl acetate (3×50 mL). The combined organic layers were washed with brine, dried over MgSO₄, filtered and concentrated under reduced pressure to yield the desired product as a white solid.

¹H NMR (400 MHz, DMSO-d₆) δ 9.19 (s, 1H), 9.04 (s, 1H), 8.74 (s, 1H), 7.65 (d, J=9.5 Hz, 1H), 7.38-7.34 (m, 1H), 7.05 (d, J=9.5 Hz, 1H), 6.64-6.60 (m, 1H), 6.50-6.48 (m, 1H), 4.58 (bs, 1H), 3.83-3.77 (m, 4H), 1.94-1.88 (m, 4H). MS (ESI, Q⁺) m/z 473, 475 (M+1 for ⁷⁹Br and ⁸¹Br).

### Example 17

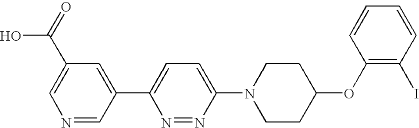

### 5-{6-[4-(2-Iodophenoxy)piperidin-1-yl]pyridazin-3-yl}nicotinic acid

### Step 1: Ethyl 5-{6-[4-(2-iodophenoxy)piperidin-1-yl]pyridazin-3-yl}nicotinate

Into a round-bottom flask equipped with a magnetic stirbar was added ethyl 5-[6-(4-hydroxypiperidin-1-yl)pyridazin-3-yl]nicotinate (230 mg, 0.700 mmol), 2-iodophenol (200 mg, 0.911 mmol), diethyl azodicarboxylate (144 μL, 0.911 mmol) and triphenylphosphine (225 mg, 0.911 mmol) in THF (1 mL). The thick suspension was sonicated for 20 min, becoming a thick orange solution. The reaction mixture was purified by column chromatography on silica gel (eluting with 40% ethyl acetate in hexanes) to afford the desired product as a white solid.

### Step 2: 5-{6-[4-(2-Iodophenoxy)piperidin-1-yl]pyridazin-3-yl}nicotinic acid

Into a round-bottom flask equipped with a magnetic stirbar and reflux condenser was added ethyl 5-{6-[4-(2-iodophenoxy)piperidin-1-yl]pyridazin-3-yl}nicotinate (1 equiv.), methanol (0.03 M) and 1 M aqueous sodium hydroxide (10 equiv.). The resulting suspension was heated to 100° C. for 2 h, and then cooled to room temperature. The reaction mixture was concentrated and poured into a separatory funnel containing pH 5 buffer and extracted three times with ethyl acetate. The combined organic layers were washed with brine, dried over MgSO₄, filtered and concentrated under reduced pressure to yield the desired product as a white solid. MS (ESI, Q⁺) m/z 503 (M+1)

### Example 18

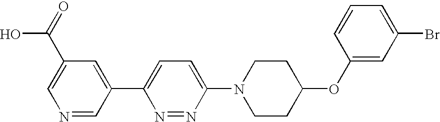

### 5-{6-[4-(3-Bromophenoxy)piperidin-1-yl]pyridazin-3-yl}nicotinic acid

### Step 1: Ethyl 5-{6-[4-(3-bromophenoxy)piperidin-1-yl]pyridazin-3-yl}nicotinate

Into a round-bottom flask equipped with a magnetic stirbar was added ethyl 5-[6-(4-hydroxypiperidin-1-yl)pyridazin-3-yl]nicotinate (1 equiv.), 3-bromophenol (1.3 equiv.), diethyl azodicarboxylate (1.3 equiv.) and triphenylphosphine (1.3 equiv.) in THF (1 M). The thick suspension was sonicated for 20 min, becoming a thick orange solution. The reaction mixture was purified by column chromatography on silica gel (eluting with 40% ethyl acetate in hexanes) to afford the desired product as a white solid.

### Step 2: 5-{6-[4-(3-Bromophenoxy)piperidin-1-yl]pyridazin-3-yl}nicotinic acid

Into a round-bottom flask equipped with a magnetic stirbar and reflux condenser was added ethyl 5-{6-[4-(3-bromophenoxy)piperidin-1-yl]pyridazin-3-yl}nicotinate (1 equiv), methanol (0.1 M) and 1 M aqueous sodium hydroxide (4.8 equiv.). The resulting suspension was heated to 100° C. for 2 h, and then cooled to room temperature. The reaction mixture was concentrated and poured into a separatory funnel containing pH 5 buffer and extracted three times with ethyl acetate. The combined organic layers were washed with brine, dried over MgSO₄, filtered and concentrated under reduced pressure to yield the desired product as a white solid.

¹H NMR (400 MHz, CD₃OD) δ 9.32 (s, 1H), 9.15 (s, 1H), 8.92 (s, 1H), 8.03-7.97 (m, 1H), 7.46-7.40 (m, 1H), 7.24-7.15 (m, 2H), 7.13-7.07 (m, 1H), 6.99 (d, J=8.0 Hz, 1H), 4.73 (bs, 1H), 4.11-4.05 (m, 2H), 3.74-3.68 (m, 2H), 2.12 (m, 2H), 1.86 (m, 2 H). MS (ESI, Q⁻) m/z 453, 455 (M−1 for ⁷⁹Br and ⁸¹Br).

### Example 19

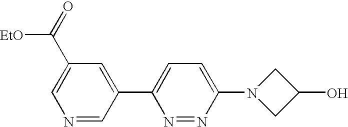

### Ethyl 5-[6-(3-hydroxyazetidin-1-yl)pyridazin-3-yl]nicotinate

### Step 1: 1-(6-Chloropyridazin-3-yl)azetidin-3-ol

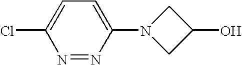

Into a round-bottom flask equipped with a reflux condenser and a magnetic stirbar was added 3,6-dichloropyridazine (1.0 equiv.), 3-hydroxyazetidine hydrochloride (1 equiv.) and water (0.9 M). The amine hydrochloride was partially neutralized with the addition of 1 M aqueous sodium hydroxide solution (0.8 equiv.). The suspension was refluxed for 48 h. The mixture was cooled and poured into a 250-mL separatory funnel containing 1 M aqueous sodium hydroxide solution (125 mL) and extracted three times with ethyl acetate. The combined organic layers were washed with brine, dried over MgSO₄, filtered and concentrated under reduced pressure. Purification by column chromatography on silica gel (eluting with 50% ethyl acetate in hexanes) gave the title compound as a light yellow solid.

### Step 2: Ethyl 5-[6-(3-hydroxyazetidin-1-yl)pyridazin-3-yl]nicotinate

Into a 15-mL Schlenk flask equipped with a magnetic stirbar was added 3-(ethoxycarbonyl)pyridine-5-boronic acid pinacol ester (858 mg, 3.10 mmol), 1-(6-chloropyridazin-3-yl)azetidin-3-ol (500 mg, 2.69 mmol), Pd₂dba₃ (25 mg, 0.03 mmol) and tricyclohexylphosphine (19 mg, 0.07 mmol). The flask was evacuated under reduced pressure and back-filled with N₂ (repeated 3 times). The solids were then treated with dioxane (6 mL) and an aqueous solution of tribasic potassium phosphate (0.972 mL, 4.58 mmol) was added. The flask was sealed and heated to 100° C. for 5 h. The reaction mixture was cooled, filtered through a sintered glass funnel containing a plug of silica gel and the solid pad washed with copious amounts of 9:1 ethyl acetate:methanol. The filtrate was concentrated under reduced pressure and purified by column chromatography through silica gel, eluting with 100% ethyl acetate.

¹H NMR (400 MHz, CDCl₃) δ 9.34 (s, 1H), 9.23 (s, 1H), 8.87 (s, 1H), 7.68 (d, J=9.5 Hz, 1H), 6.67 (d, J=9.5 Hz, 1H), 4.94 (bs, 1H), 4.53-4.40 (m, 5 H), 4.12 (dd, J=9.5, 4.5 Hz, 2H), 3.49 (bs, 1H), 1.44 (t, J=7.0 Hz, 3H). MS (ESI, Q⁻) m/z 301 (M+1).

### Example 20

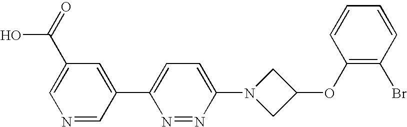

### 5-{6-[3-(2-Bromophenoxy)azetidin-1-yl]pyridazin-3-yl}nicotinic acid

### Step 1: Ethyl 5-{6-[3-(2-bromophenoxy)azetidin-1-yl]pyridazin-3-yl}nicotinate

Into a round-bottom flask equipped with a magnetic stirbar, a reflux condenser and under N₂ was added ethyl 5-[6-(3-hydroxyazetidin-1-yl)pyridazin-3-yl]nicotinate (1 equiv.), 2-bromophenol (1.2 equiv.), 1,1′-(azodicarbonyl)dipiperidine (1.2 equiv.) and THF (0.2 M). The suspension was heated to 80° C. and then tri-n-butylphosphine (1.2 equiv.) was added dropwise and the solution heated for 16 h. The cooled reaction mixture was quenched with 25 mL of 1M aqueous hydrochloric acid and stirred at room temperature for 30 min. The reaction was basified to pH=9 and then poured into a separatory funnel containing water and the mixture was extracted three times with ethyl acetate. The combined organic layers were washed with brine, dried over MgSO₄, filtered and concentrated under reduced pressure. Purification by column chromatography on silica gel (eluting with 40% ethyl acetate in hexanes) gave the title compound as a white solid.

### Step 2: 5-{6-[3-(2-Bromophenoxy)azetidin-1-yl]pyridazin-3-yl}nicotinic acid

Into a round-bottom flask equipped with a magnetic stirbar was added ethyl 5-{6-[3-(2-bromophenoxy)azetidin-1-yl]pyridazin-3-yl}nicotinate (1.0 equiv.), methanol (0.1 M) and 1 M aqueous sodium hydroxide (5.0 equiv.). The resulting suspension was heated to 100° C. for 2 h, and then cooled to room temperature. The reaction mixture was concentrated and poured into a separatory funnel containing pH=5 buffer and extracted three times with ethyl acetate. The combined organic layers were washed with brine, dried over MgSO₄, filtered and concentrated under reduced pressure to yield the desired product as a white solid.

¹H NMR (400 MHz, CD₃OD) δ 9.33 (s, 1H), 9.16 (s, 1H), 8.93 (s, 1H), 8.04 (d, J=9.5 Hz, 1H), 7.60 (d, J=8.0 Hz, 1H), 7.38-7.32 (m, 1H), 7.06 (d, J=9.5 Hz, 1H), 6.98-6.87 (m, 2H), 5.36-5.30 (m, 1H); 4.70 (dd, J=9.5, 6.5 Hz, 2H), 4.28 (dd, J=9.5, 4.0 Hz, 2H). MS (ESI, Q⁻) m/z 425, 427 (M−1 for ⁷⁹Br and ⁸¹Br).

### Example 21

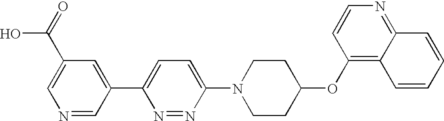

### 5-{6-[4-(Quinolin-4-yloxy)piperidin-1-yl]pyridazin-3-yl}nicotinic acid

### Step 1: Ethyl 5-{6-[4-(quinolin-4-yloxy)piperidin-1-yl]pyridazin-3-yl}nicotinate

Into a round-bottom flask equipped with a magnetic stirbar was added ethyl 5-[6-(4-hydroxypiperidin-1-yl)pyridazin-3-yl]nicotinate (1 equiv.), 4-hydroxyquinoline (1.3 equiv.), diethyl azodicarboxylate (1.3 equiv.) and triphenylphosphine (1.3 equiv.) in THF (1 M). The thick suspension was sonicated for 20 min, becoming a thick orange solution. The reaction mixture was purified by column chromatography on silica gel (eluting with 100% ethyl acetate) to afford the desired product as a white solid.

### Step 2: 5-{6-[4-(Quinolin-4-yloxy)piperidin-1-yl]pyridazin-3-yl}nicotinic acid

Into a round-bottom flask equipped with a magnetic stirbar was added ethyl 5-{6-[4-(quinolin-4-yloxy)piperidin-1-yl]pyridazin-3-yl}nicotinate (1 equiv.), methanol (0.1 M) and 1 M aqueous sodium hydroxide (4.5 equiv.). The resulting suspension was heated to 100° C. for 2 h, and then cooled to room temperature. The reaction mixture was concentrated and poured into a separatory funnel containing pH 5 buffer and extracted three times with ethyl acetate. The combined organic layers were washed with brine, dried over MgSO₄, filtered and concentrated under reduced pressure to yield the desired product as a white solid.

¹H NMR (400 MHz, CD₃OD) δ 9.21 (s, 1H), 9.12 (s, 1H), 8.83 (s, 1H), 8.70 (d, J=5.5 Hz, 1 H), 8.31 (d, J=8.5 Hz, 1H), 7.99-7.94 (m, 2H), 7.76 (t, J=7.5 Hz, 1H), 7.58 (t, J=7.5 Hz, 1H), 7.47 (d, J=9.5 Hz, 1H), 7.14 (d, J=5.5 Hz, 1H), 5.16-5.10 (m, 1H), 4.18-4.10 (m, 2 H), 3.88-3.80 (m, 2H), 2.33-2.25 (m, 2H), 2.13-2.03 (m, 2H). MS (ESI, Q⁺) m/z 456 (M+1).

### Example 22

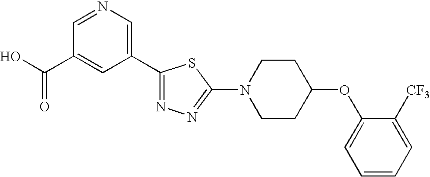

### 5-[5-(4-{[2-(Trifluoromethyl)phenyl]oxy}piperidin-1-yl)-1,3,4-thiadiazol-2-yl]pyridine-3-carboxylic acid

### Step 1: 5-{4-[2-(Trifluoromethyl)phenoxy]piperidin-1-yl}-1,3,4-thiadiazol-2-amine

To a solution of 4-[2-(trifluoromethyl)phenoxy]piperidine hydrochloride (1 equiv.) in DMF (0.04 M) was added 5-bromo-1,3,4-thiadiazol-2-amine (1 equiv.) and K₂CO₃ (3 equiv.). The reaction was heated at 80° C. with stirring overnight. After cooling, the salt was removed by filtration and the filtrate was evaporated in vacuo. The residue was washed with ethyl acetate to afford the title compound.

¹H NMR (400 MHz, DMSO-d₆): δ 7.57-7.60 (m, 2H), 7.29-7.35 (m, 1H), 7.03-7.05 (m, 1H), 6.46 (s, 2H), 4.84 (s, 1H), 3.22-3.30 (m, 4H), 1.91-2.01 (m, 2H), 1.68-1.78 (m, 2H). MS: m/z 345 (MH⁺).

### Step 2: 1-(5-Bromo-1,3,4-thiadiazol-2-yl)-4-[2-(trifluoromethyl)phenoxy]piperidine

To a suspension of 5-{4-[2-(trifluoromethyl)phenoxy]piperidin-1-yl}-1,3,4-thiadiazol-2-amine (1 equiv.) in acetonitrile (0.03 M) was added CuBr₂ (2 equiv.). After 5 min, t-butyl nitrite (2 equiv.) was added and the reaction mixture stirred at room temperature for 15 min. The reaction mixture was then heated at 50-60° C. until TLC indicated complete consumption of starting material. The solvent was evaporated, and EtOAc and water were added. The solid was removed by filtration and the filtrate was extracted three times with EtOAc and dried over anhydrous Na₂SO₄ Solvents were removed in vacuo to afford the crude product, which was purified by Combiflash (SiO₂, gradient elution 20-50% EtOAc/hexanes) to yield the title compound as a solid.

¹H NMR (400 MHz, acetone-d₆): δ 7.65-7.57 (m, 2H), 7.34 (d, 1H), 7.09 (t, 1H), 5.01-4.96 (m, 1H), 3.72 (ddd, 2H), 3.66-3.58 (m, 2H), 2.20-2.11 (m, 2H), 2.03-1.95 (m, 2H). MS: m/z 408, 410 (MH⁺).

### Step 3: Ethyl-5-[5-(4-{[2-(trifluoromethyl)phenyl]oxy}piperidin-1-yl)-1,3,4-thiadiazo-1-2-yl]pyridine-3-carboxylate

A mixture of 1-(5-bromo-1,3,4-thiadiazol-2-yl)-4-[2-(trifluoromethyl)phenoxy]piperidine (1 equiv.), ethyl-5-(4,4,5,5-tetramethyl-1,3,2-dioxaborolan-2-yl)pyridine-3-carboxylate (1.2 equiv.), Pd(dppf)Cl₂ (0.15 equiv.) and 2M aqueous Na₂CO₃ (2.3 equiv.) in DMF (0.2 M) was degassed for 5 min with N₂ gas. The mixture was then heated at 80° C. After 5 h, the mixture was cooled to room temperature, diluted with EtOAc, and filtered through celite. The filtrate was diluted with water and extracted three times with EtOAc. The combined organic extracts were washed with water (25 mL) and dried over anhydrous Na₂SO₄. Solvents were removed in vacuo to afford the crude product, which was purified by Combiflash (SiO₂, gradient elution 50-80 % MeOH/EtOAc) to yield the title compound as a solid.

¹H NMR (500 MHz, acetone-d₆): δ 9.16 (s, 1H), 9.13 (s, 1H), 8.62 (t, 1H), 7.66-7.58 (m, 2 H), 7.36 (d, 1H), 7.10 (t, 1H), 5.05-5.01 (m, 1H), 4.43 (q, 2H), 3.88-3.81 (m, 2H), 3.79-3.72 (m, 2H), 2.24-2.17 (m, 2H), 2.05-2.01 (m, 2H), 1.40 (t, 3H).MS: m/z 479 (MH⁺).

### Step 4: 5-[5-(4-{[2-(trifluoromethyl)phenyl]oxy}piperidin-1-yl)-1,3,4-thiadiazol-2-yl]pyridine-3-carboxylic acid

To a solution of ethyl-5-[5-(4-{[2-(trifluoromethyl)phenyl]oxy}piperidin-1-yl)-1,3,4-thiadiazo-1-2-yl]pyridine-3-carboxylate (1 equiv.) in THF (0.2 M) was added 2 M aqueous NaOH (10 equiv.). The mixture was heated at 60° C. for 2 h, the THF was removed, and the aqueous residue was then washed twice with EtOAc. The aqueous layer was acidified to pH about 5 with 2N HCl. The solid precipitate was slurried with Et₂O, filtered, and washed with water and then Et₂O to give the title compound as a solid.

¹H NMR (500 MHz, acetone-d₆): δ 9.15 (2 x d, 2H), 8.65 (s, 1H), 7.62 (d, 2H), 7.37 (d, 1H), 7.10 (s, 1H), 4.97 (br s, 1H), 3.83 (dd, 2H), 3.78-3.71 (m, 2H), 2.25-2.16 (m, 2H), 2.02-1.97 (m, 2H). MS: m/z 451 (MH⁺).

### Example 23

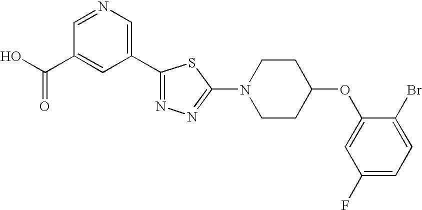

### 5-{5-[4-(2-bromo-5-fluorophenoxy)piperidin-1-yl]-1,3,4-thiadiazol-2-yl}nicotinic acid

### Step 1: 1-(5-amino-1,3,4-thiadiazol-2-yl)piperidin-4-ol

The title compound was prepared in the same manner as described in Example 22 (step 1) from piperidin-4-ol and 5-bromo-1,3,4-thiadiazol-2-amine. MS: m/z 201 (MH⁺).

### Step 2: 1-(5-bromo-1,3,4-thiadiazol-2-yl)piperidin-4-ol

The title compound was prepared in the same manner as described in Example 22 (step 2) from 1-(5-amino-1,3,4-thiadiazol-2-yl)piperidin-4-ol, CuBr₂ and t-butyl nitrite. MS: m/z 264, 266 (MH⁺).

### Step 3: Ethyl 5-[5-(4-hydroxypiperidin-1-yl)-1,3,4-thiadiazol-2-yl]nicotinate

The title compound was prepared in the same manner as described in Example 22 (step 3) from 1-(5-bromo-1,3,4-thiadiazol-2-yl)piperidin-4-ol, 5-bromo-1,3,4-thiadiazol-2-amine, ethyl-5-(4,4,5,5-tetramethyl-1,3,2-dioxaborolan-2-yl)pyridine-3-carboxylate and Pd(dppf)Cl₂. MS: m/z 335 (MH⁺).

### Step 4: Ethyl-5-{5-[4-(2-bromo-5-fluorophenoxy)piperidin-1-yl]-1,3,4-thiadiazol-2-yl}nicotinic acid

The title compound was prepared in the same manner as described in Example 15 from ethyl 5-[5-(4-hydroxypiperidin-1-yl)-1,3,4-thiadiazol-2-yl]nicotinate -bromo-5-fluorophenol, diethyl azodicarboxylate and triphenylphosphine. MS: m/z 507, 509 (MH⁺).

### Step 5: 5-{5-[4-(2-Bromo-5-fluorophenoxy)piperidin-1-yl]-1,3,4-thiadiazol-2-yl-nicotinic acid

The title compound was prepared in the same manner as described in Example 22 (step 4) from ethyl-5-{5-[4-(2-bromo-5-fluorophenoxy)piperidin-1-yl]-1,3,4-thiadiazol-2-yl-nicotinic acid and 2 M aqueous NaOH.

¹H NMR (500 MHz, acetone-d₆): δ 9.19 (s, 1H), 9.16 (s, 1H), 8.66 (s, 1H), 7.60 (dd, 1H), 7.12 (dd, 1H), 6.75 (td, 1H), 4.97 (d, 1H), 3.94-3.88 (m, 2H), 3.80-3.74 (m, 2H), 2.24-2.17 (m, 2H), 2.04-2.00 (m, 2H). MS: m/z 479, 481 (MH⁺).

### Example 24

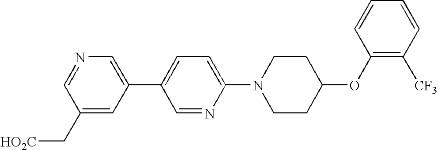

### (6′-{4-[2-(Trifluoromethyl)phenoxy]piperidin-1-yl}-3,3′-bipyridin-5-yl)acetic acid

### Step 1: Methyl(5-bromopyridin-3-yl)acetate

To a solution of (5-bromopyridin-3-yl)acetic acid in methanol (0.1 M) was added a slight excess of a solution of diazomethane in ether at room temperature. After stirring for 5 min, solvents and residual diazomethane were removed under reduced pressure to afford the title compound.

### Step 2: (6′-{4-[2-(trifluoromethyl)phenoxy]piperidin-1-yl}-3,3′-bipyridin-5-yl)acetic acid

To a solution of methyl(5-bromopyridin-3-yl)acetate (1 equiv.) and 5-(4,4,5,5-tetramethyl-1,3,2-dioxaborolan-2-yl)-2-{4-[2-(trifluoromethyl)phenoxy]piperidin-1-yl}pyridine (prepared as described in example 1, step 3) (1.4 equiv.) in DMF (0.045 M), 2N aqueous sodium carbonate solution (3.3 equiv.) and tetrakis(triphenylphosphine)palladium(0) (0.1 equiv.) were added. The reaction mixture was then stirred overnight at 100° C. After cooling, the mixture was diluted with water and washed with ethyl acetate. The aqueous layer was then acidified with a saturated NH₄Cl aqueous solution and the product was extracted twice with ethyl acetate. The combined organic layers were dried over MgSO₄, filtered and concentrated to afford the title compound. MS: m/z 458.1 (ESI+).

### Example 25

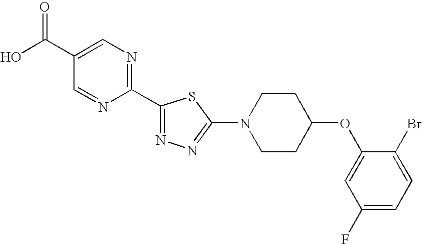

### 2-{5-[4-(2-Bromo-5-fluorophenoxy)piperidin-1-yl]-1,3,4-thiadiazol-2-yl}pyrimidine-5-carboxylic acid

### Step 1: 4-(2-Bromo-5-fluorophenoxy)piperidine

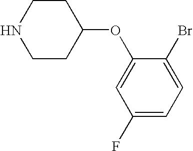

To a solution of tert-butyl 4-hydroxypiperidine-1-carboxylate in dichloromethane (0.5 M) was added MsCl (1.2 equiv) and Et₃N (1.7 equiv) at 0° C. The mixture was further stirred for 3 h and filtered. The filtrate was evaporated in vacuo to give tert-butyl 4-[(methylsulfonyl)oxy]piperidine-1-carboxylate. ¹H NMR (400 MHz, CDCl₃) δ 4.84-4.91 (m, 1H), 3.64-3.75 (m, 2H), 3.24-3.35 (m, 2H), 3.04 (s, 3H), 1.91-2.02 (m, 2H), 1.76-1.87 (m, 2H), 1.48 (s, 9 H). MS: m/z 280 (MH⁺).

A solution of tert-butyl 4-[(methylsulfonyl)oxy]piperidine-1-carboxylate in DMF (1.0 M) was added 2-bromo-5-fluorophenol (1.2 equiv) and Cs₂CO₃ (2.0 equiv). The reaction mixture was heated at 70° C. overnight. The solvent was evaporated in vacuo, and the residue was purified by column chromatography to give tert-butyl 4-(2-bromo-5-fluorophenoxy)piperidine-1-carboxylate. The product was used directly in next step without purification.

A solution of tert-butyl 4-(2-bromo-5-fluorophenoxy)piperidine-1-carboxylate in ethanol (4.5 M) was added dropwise 5 N HCl in ethanol solution (1.3 equiv). The reaction mixture was stirred at room temperature for 12 h. The solvent was evaporated in vacuo, and ether was added to the residue. The resulting precipitate was washed with ether to afford the title compound in the form of its hydrochloride salt. ¹H NMR (300 MHz, D₂O): δ 7.44-7.49 (m, 1H), 6.83-6.88 (m, 1H), 6.50-6.67 (m, 1H), 4.67-4.73 (m, 1H), 3.30-3.39 (m, 2H), 3.13-3.23 (m, 2H), 2.03-2.08 (m, 4H).

The salt was neutralized with 1 N aqueous NaOH, extracted with EtOAc, washed with brine, dried over Na₂SO₄, filtered and concentrated under reduced pressure. The title compound was used directly in step 3 without further purification.

### Step 2: 5-Bromo-1,3,4-thiadiazole-2-carbonitrile

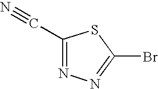

To a suspension of 5-bromo-1,3,4-thiadiazol-2-amine and cuprous cyanide (2.2 equiv) in acetonitrile (0.3 M) at 0° C. was added dropwise t-BuONO (2.1 equiv) over 20 min. The suspension was stirred at room temperature until TLC showed that the reaction was completed. The reaction mixture was then filtered and the filtrate was concentrated in vacuo to give the crude product which was purified by chromatography to give the title product. ¹³C NMR (300 MHz, CDCl₃): δ 77.3, 109.0, 141.7.

### Step 3: 5-[4-(2-Bromo-5-fluorophenoxy)piperidin-1-yl]-1,3,4-thiadiazole-2-carbonitrile

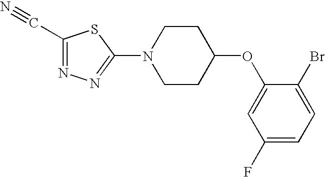

To a solution of 4-(2-bromo-5-fluorophenoxy)piperidine in 1,4-dioxane (0.6 M) was added Hunig's base (3.3 equiv) followed by 5-bromo-1,3,4-thiadiazole-2-carbonitrile (1.0 equiv). The final mixture was stirred 1 h at room temperature. The reaction mixture was purified by column chromatography on silica gel (eluting with 10-40% ethyl acetate in hexanes) to afford the desired product as a colorless oil.

¹H NMR (500 MHz, acetone-d₆) δ 7.63 (dd, 1H), 7.14 (dd, 1H), 6.78 (td, 1H), 5.04-4.99 (m, 1H), 4.00-3.95 (m, 2H), 3.89-3.84 (m, 2H), 2.27-2.21 (m, 2H), 2.11-2.05 (m, 2H).

### Step 4: Methyl 2-{5-[4-(2-bromo-5-fluorophenoxy)piperidin-1-yl]-1,3,4-thiadiazol-2-yl}pyrimidine-5-carboxylate

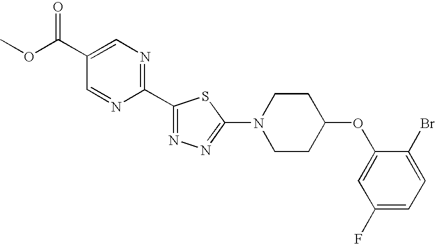

To a solution of 5-[4-(2-bromo-5-fluorophenoxy)piperidin-1-yl]-1,3,4-thiadiazole-2-carbonitrile in DMF (0.4 M) was added LiHMDS (1.0 M in hexanes, 1.0 equiv). After 15 min, pyridinium hydrochloride (2.1 equiv) was added to the reaction mixture followed by the sodium salt of 3,3-dimethoxy-2-methoxycarbonylpropen-1-ol (Zhichkin, P.; Fairfax, D. J.; Eisenbeis S. A. Synthesis 2002, 720-722) (1.6 equiv). The final mixture was heated to 100° C. for 1.5 h. The reaction mixture was poured into 0.5 N aqueous HCl, extracted with EtOAc, washed with brine, dried over Na₂SO₄, filtered and concentrated under reduced pressure. The crude material was purified by column chromatography on silica gel (eluting with 10-75% ethyl acetate in hexanes) to afford the desired product as a yellow solid.

¹H NMR (400 MHz, acetone-d₆) δ 9.29 (s, 2H), 7.64 (dd, 1H), 7.15 (dd, 1H), 6.78 (td, 1H), 5.04-4.98 (m, 1H), 4.02 (s, 3H), 4.02-3.94 (m, 2H), 3.89-3.83 (m, 2H), 2.28-2.21 (m, 2H), 2.11-2.03 (m, 2H). MS: m/z 496,494 (MH⁺).

### Step 5: 2-{5-[4-(2-Bromo-5-fluorophenoxy)piperidin-1-yl]-1,3,4-thiadiazol-2-yl}pyrimidine-5-carboxylic acid

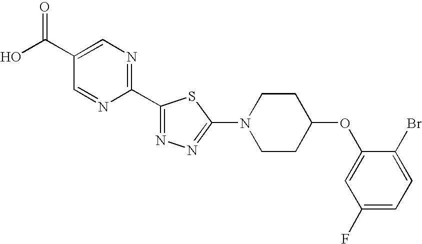

To a solution of methyl 2-{5-[4-(2-bromo-5-fluorophenoxy)piperidin-1-yl]-1,3,4-thiadiazol-2-yl}pyrimidine-5-carboxylate in THF:MeOH (2:1) (v/v) (0.03 M) was added 1.0 N aqueous NaOH (12 equiv). The final mixture was stirred 10 min at room temperature. The reaction mixture was poured into 0.5 N aqueous HCl, extracted with EtOAc, washed with brine, dried over Na₂SO₄, filtered and concentrated under reduced pressure. The crude product was triturated with Et₂O/heptane to afford the title compound as a yellow solid.

¹H NMR (500 MHz, acetone-d₆) δ 9.31 (s, 2H), 7.64 (dd, 1H), 7.16 (dd, 1H), 6.78 (m, 1H), 5.01 (m, 1H), 4.00-3.95 (m, 2H), 3.88-3.84 (m, 2H), 2.27-2.21 (m, 2H), 2.10-2.04 (m, 2H). MS: m/z 482, 480 (MH+).

### Example 26

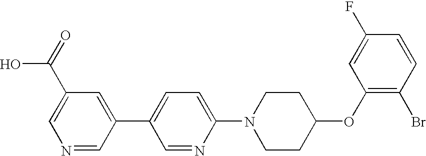

### 6′-[4-(2-Bromo-5-fluorophenoxy)piperidin-1-yl]-3,3′-bipyridine-5-carboxylic acid

### Step 1: Ethyl 6′-(4-hydroxypiperidin-1-yl)-3,3′-bipyridine-5-carboxylate

A mixture of 1-(5-bromo-2-pyridinyl)-4-piperidinol [from Example 1, step 1], 3-(ethoxycarbonyl)pyridine-5-boronic acid pinacol ester (1.15 equiv.), Cs₂CO₃ (2.0 equiv.) and palladium(I) tri-tertbutylphosphine bromide dimer (0.02 equiv.) in DMF (0.5 M) was heated at 120° C. for 1 h. The reaction mixture was partitioned between ethyl acetate and water. The organic phase was separated, dried over Na₂SO₄ and evaporated. The title compound was purified over silica gel eluting with ethyl acetate to 5% methanol in ethyl acetate.

### Step 2: Ethyl 6′-[4-(2-bromo-5-fluorophenoxy)piperidin-1-yl]-3,3′-bipyridine-5-carboxylate

The title compound was prepared from ethyl 6′-(4-hydroxypiperidin-1-yl)-3,3′-bipyridine-5-carboxylate and 2-bromo-5-fluorophenol as described in Step 1 of Example 21.

### Step 3: 6′-[4-(2-Bromo-5-fluorophenoxy)piperidin-1-yl]-3,3′-bipyridine-5-carboxylic acid

The title compound was prepared from ethyl 6′-[4-(2-bromo-5-fluorophenoxy)piperidin-1-yl]-3,3′-bipyridine-5-carboxylate as described in Step 5 of Example 1, except that after hydrolysis, the reaction mixture was partitioned between ethyl acetate and ammonium chloride. The organic solvent was separated, dried over Na₂SO₄, filtered and evaporated. Ether and ethyl acetate were added to provide a solid which was then collected by filtration. MS: m/z 472.0 (ESI +).

### Example 27

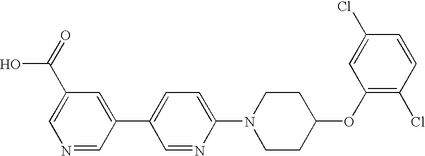

### 6′-[4-(2,5-Dichlorophenoxy)piperidin-1-yl]-3,3′-bipyridine-5-carboxylic acid

The title compound was prepared as described in Example 26. MS: m/z 444.0 (ESI+)

### Example 28

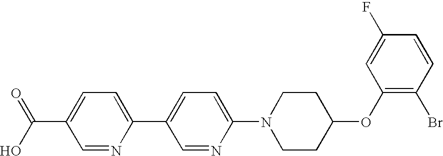

### 6′-[4-(2-Bromo-5-fluorophenoxy)piperidin-1-yl]-2,3′-bipyridine-5-carboxylic acid

### Step 1: 5-Bromo-2-(4-{[tert-butyl(dimethyl)silyl]oxy}piperidin-1-yl)pyridine

To a solution of 1-(5-bromo-2-pyridinyl)-4-piperidinol [from example 1 step 1] and imidazole (1.5 equiv.) in DMF (0.5 M) was added tert-butyldimethylsilyl chloride (1.3 equiv.) followed by a catalytic amount of DMAP. After a period of 2 h, the reaction mixture was partitioned between ethyl acetate and water. The organic solvent was separated, dried over Na2SO4, filtered and evaporated. The title compound was purified by flash chromatography eluting with 10% ethyl acetate in hexane.

### Step 2: 2-(4-{[tert-Butyl(dimethyl)silyl]oxy}piperidin-1-yl)-5-(4,4,5,5-tetramethyl-1,3,2-dioxaborolan-2-yl)pyridine

The title compound was prepared from 5-bromo-2-(4-{[tert-butyl(dimethyl)silyl]oxy}piperidin-1-yl)pyridine as described in Step 3 of example 1.

### Step 3: Methyl 6′-(4-{[tert-butyl(dimethyl)silyl]oxy}piperidin-1-yl)-2,3′-bipyridine-5-carboxylate

A mixture of methyl 6-bromonicotinate, 2-(4-{[tert-butyl(dimethyl)silyl]oxy}piperidin-1-yl)-5-(4,4,5,5-tetramethyl-1,3,2-dioxaborolan-2-yl)pyridine (1.1 equiv.), tetrakis(triphenylphosphine)palladium(0) (0.04 equiv.), 2 M sodium carbonate (2.5 equiv.) in DMF (0.1 M) was purged twice with nitrogen and heated at reflux for 18 h. After cooling, the reaction mixture was partitioned between ethyl acetate and saturated ammonium chloride. The organic solvent was separated, dried over magnesium sulfate, filtered and evaporated. The crude product was then purified by flash chromatography.

### Step 4: Methyl 6′-(4-hydroxypiperidin-1-yl)-2,3′-bipyridine-5-carboxylate

To a solution of methyl 6′-(4-{[tert-butyl(dimethyl)silyl]oxy}piperidin-1-yl)-2,3′-bipyridine-5-carboxylate in THF (0.09 M) was added TBAF (1.1 equiv.). After a period of 2 h, the reaction mixture was partitioned between ethyl acetate and saturated ammonium acetate. The organic solvent was separated, dried over magnesium sulfate, filtered and evaporated. The title compound was purified by flash chromatography eluting with 2% methanol in ethyl acetate.

### Step 5: Methyl 6′-[4-(2-bromo-5-fluorophenoxy)piperidin-1-yl]-2,3′-bipyridine-5-carboxylate

The title compound was prepared from methyl 6′-(4-hydroxypiperidin-1-yl)-2,3′-bipyridine-5-carboxylate and 2-bromo-5-fluorophenol as described in Step 1 of example 21.

### Step 6: 6′-[4-(2-Bromo-5-fluorophenoxy)piperidin-1-yl]-2,3′-bipyridine-5-carboxylic acid

To a mixture of methyl 6′-[4-(2-bromo-5-fluorophenoxy)piperidin-1-yl]-2,3′-bipyridine-5-carboxylate in MeOH-THF (1:1) (0.06M) was added a 1 M aqueous solution of lithium hydroxide (4.4 equiv.). After a period of 2 h at reflux, the solvents were removed under reduced pressure. To the residue was added water and ethyl acetate. The water was then collected followed by the addition of ethyl acetate and saturated aqueous ammonium chloride. The organic phase was separated, dried over MgSO₄, filtered and evaporated. MS: m/z 472.3 (ESI+).

### Example 29

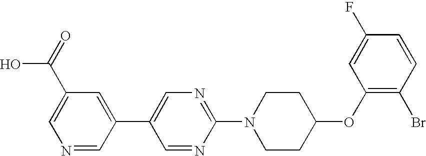

### 5-{2-[4-(2-Bromo-5-fluorophenoxy)piperidin-1-yl]pyrimidin-5-yl}nicotinic acid

### Step 1: 1-(5-Bromopyrimidin-2-yl)piperidin-4-ol

To a mixture of 5-bromo-2-chloropyrimidine and 4-hydroxypiperidine (2.4 equiv.) in 2-propanol (0.5 M) was added N,N-diisopropylethylamine (1.7 equiv.). After a period of 5 min in microwave at 160° C., the reaction mixture was partitioned between ethyl acetate and aqueous sodium carbonate. The organic phase was separated, dried over Na₂SO₄, filtered and evaporated. To the residue was added 5% ether in hexane to produce a beige solid which was collected by filtration.

### Step 2: Ethyl 3-[2-(4-hydroxypiperidin-1-yl)pyrimidin-5-yl]benzoate

A mixture of 3-(ethoxycarbonyl)pyridine-5-boronic acid pinacol ester, 1-(5-bromopyrimidin-2-yl)piperidin-4-ol (0.7 equiv.), Pd₂(dba)₃ (0.01 equiv.), tricyclohexylphosphine (0.02 equiv.) and tri-potassium phosphate (1.13 equiv.) in dioxane-water (5:1) (0.6 M) was purged with nitrogen and heated at 100° C. After a period of 2 h, the reaction mixture was partitioned between ethyl acetate and water. The organic solvent was separated, dried over Na₂SO₄, filtered and evaporated. The title product was purified by flash chromatography eluting with pure ethyl acetate to give a white solid.

### Step 3: Ethyl 3-{2-[4-(2-bromo-5-fluorophenoxy)piperidin-1-yl]pyrimidin-5-yl}benzoate

The title compound was prepared, as described in Step 1 of example 21, from ethyl 3-[2-(4-hydroxypiperidin-1-yl)pyrimidin-5-yl]benzoate and 2-bromo-5-fluorophenol.

### Step 4: 5-{2-[4-(2-Bromo-5-fluorophenoxy)piperidin-1-yl]pyrimidin-5-yl}nicotinic acid

To a mixture of ethyl 3-{2-[4-(2-bromo-5-fluorophenoxy)piperidin-1-yl]pyrimidin-5-yl}benzoate in THF-MeOH (2:1) (0.07 M) was added 1 M sodium hydroxide (5 equiv.). After a period of 1 h, the reaction mixture was partitioned between ethyl acetate and saturated aqueous ammonium chloride. The organic solvent was separated, dried over Na₂SO₄, filtered and evaporated. To the residue was added ether and the resulting solid was collected. MS: m/z 472.9 (ESI +).

### Example 30

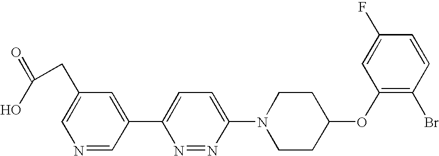

### (5-{6-[4-(2-Bromo-5-fluorophenoxy)piperidin-1-yl]pyridazin-3-yl}pyridin-3-yl)acetic acid

### Step 1: Methyl [5-(4,4,5,5-tetramethyl-1,3,2-dioxaborolan-2-yl)pyridin-3-yl]acetate

A mixture of methyl(5-bromopyridine-3-yl)acetate [from example 24, step 1], bis(pinacolato)diboron (1.25 equiv.), potassium acetate (3 equiv.), palladium (II) dichloride (dppf) (0.1 equiv.) in DMF (0.4 M) was heated, under nitrogen, at 100° C. for 18 h. The reaction mixture was then filtered over celite and washed with dichloromethane. The filtrate was concentrated under reduced pressure and the crude residue was taken in heptane. The solid was filtered off and the filtrate containing the title compound was concentrated without further purification.

### Step 2: 1-(6-Bromopyridazin-3-yl)piperidin-4-ol

A suspension of 3,6-dibromopyridazine and 4-hydroxypiperidine (1.5 equiv.) in isopropanol (2 M) was heated in microwave at 150° C. After a period of 20 min., the crude residue was partitioned between ethyl acetate and water. The organic phase was separated, dried over MgSO₄, filtered and evaporated under reduced pressure. The title compound was purified by flash chromatography eluting with 50% acetone in dichloromethane.

### Step 3: Methyl {5-[6-(4-hydroxycyclohexyl)pyridazin-3-yl]pyridin-3-yl}acetate

The title compound was prepared as described in Step 2 of example 29 from methyl [5-(4,4,5,5-tetramethyl-1,3,2-dioxaborolan-2-yl)pyridin-3-yl]acetate and 1-(6-bromopyridazin-3-yl)piperidin-4-ol , except that the reaction was heated for 18 h.

### Step 4: Methyl(5-{6-[4-(2-bromo-5-fluorophenoxy)cyclohexyl]pyridazin-3-yl}pyridin-3-yl)acetate

The title compound was prepared as described in Step 1 of example 21 from methyl {5-[6-(4-hydroxycyclohexyl)pyridazin-3-yl]pyridin-3-yl}acetate and 2-bromo-5-fluorophenol.

### Step 5: 5-{6-[4-(2-Bromo-5-fluorophenoxy)piperidin-1-yl]pyridazin-3-yl}pyridin-3-yl)acetic acid

A solution of methyl(5-{6-[4-(2-bromo-5-fluorophenoxy)cyclohexyl]pyridazin-3-yl}pyridin-3-yl)acetate in methanol-THF(1:2) (0.08 M) was treated with 1 M LiOH (3.0 equiv.). After a period of 1 h at reflux, the solvents were removed under reduced pressure and a mixture of ether-hexane (1:1) was added. Saturated aqueous ammonium chloride was added followed by ethyl acetate. The organic phase was separated, dried over MgSO₄, filtered and evaporated under reduced pressure. A mixture of hexane-ethyl acetate was added to the solid which was then collected by filtration. MS: m/z 487.0 (ESI +).

### Example 31

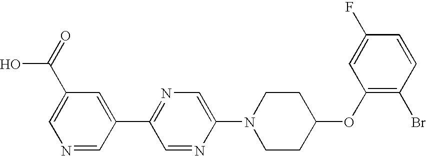

### 5-{5-[4-(2-Bromo-5-fluorophenoxy)piperidin-1-yl]pyrazin-2-yl}nicotinic acid

### Step 1: 2,5-Dichloropyrazine

A mixture of 2-hydroxy-5-chloropyrazine in POCl₃ (21 equiv.) was heated at 120° C. for 2 h. The reaction mixture was cooled and poured on ice and extracted with dichloromethane. The organic solvent was collected, dried over Na₂SO₄ and filtered. The solvent was filtered over silica gel followed by ethyl acetate. The solvents were evaporated to provide the title compound.

### Step 2: 1-(5-Chloropyrazin-2-yl)piperidin-4-ol

To a mixture of 2,5-dichloropyrazine in 2-propanol (0.2 M) was added 4-hydroxypiperidine (2.2 equiv.). The reaction was heated in the microwave at 160° C. for 10 min. The solvent was evaporated under reduced pressure and the title compound was purified by flash chromatography eluting with ethyl acetate.

### Step 3: Ethel 5-[5-(4-hydroxypiperidin-1-yl)pyrazin-2-yl]nicotinate

The title compound was prepared, as described in Step 2 of example 29, from 1-(5-chloropyrazin-2-yl)-piperidin-4-ol.

### Step 4: Ethyl 5-{5-[4-(2-bromo-5-fluorophenoxy)piperidin-1-yl]pyrazin-2-yl}nicotinate

The title compound was prepared, as described in Step 1 of example 21, from ethyl 5-[5-(4-hydroxypiperidin-1-yl)pyrazin-2-yl]nicotinate and 2-bromo-5-fluorophenol.

### Step 5: 5-{5-[4-(2-Bromo-5-fluoro-phenoxy)piperidin-1-yl]pyrazin-2-yl}nicotinic acid

The title compound was prepared, as described in Step 4 of example 29, from ethyl 5-{5-[4-(2-bromo-5-fluorophenoxy)piperidin-1-yl]pyrazin-2-yl}nicotinate, except that ethyl acetate was added to the solid which was collected by filtration. MS: m/z 473.0 (ESI +).

### Example 32

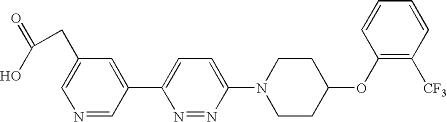

### (5-{6-[4-(2-Trifluoromethylphenoxy)piperidin-1-yl]pyridazin-3-yl}pyridin-3-yl)acetic acid

The title compound was prepared as described in example 30 using 2-trifluoromethylphenol for the Mitsunobu reaction. MS: 473.2 (ESI +).

### Example 33

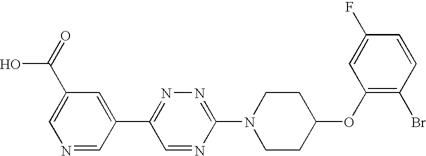

5-{3-[4-(2-Bromo-5-fluorophenoxy)piperidin-1-yl]-1,2,4-triazin-6-yl}nicotinic acid

### Step 1: 3-Amino-6-bromo-1,2,4-triazine

To 3-amino-1,2,4-triazine in a mixture of methanol-water (2:1) (1.6 M) was slowly added bromine (1.0 equiv.). After a period of 1 h at room temperature, the solvent was removed under reduced pressure. The crude mixture was partitioned between ethyl acetate and saturated sodium bicarbonate. The organic phase was separated, dried over Na₂SO₄, filtered and evaporated. Ether was added to the residue and the resulting solid filtered.

### Step 2: Ethyl 5-(3-amino-1,2,4-triazin-6-yl)nicotinate

The title compound was prepared, as described in Step 2 of example 29, using 3-amino-6-bromo-1,2,4-triazine.

### Step 3: Ethyl 5-(3-bromo-1,2,4-triazin-6-yl)nicotinate

To ethyl 5-(3-amino-1,2,4-triazin-6-yl)nicotinate in bromoform (0.1 M) at 80° C. was added isoamyl nitrite (3.2 equiv.). The resulting mixture was then heated at 85° C. for 0.5 h. The reaction mixture was evaporated under reduced pressure and purified by flash chromatography eluting with 50% ethyl acetate in hexane.

### Step 4: Ethyl 5-{3-[4-(2-bromo-5-fluorophenoxy)piperidin-1-yl]-1,2,4-triazin-6-yl}nicotinate

To a mixture of ethyl 5-(3-bromo-1,2,4-triazin-6-yl)nicotinate and 4-(2-bromo-5-fluorophenoxy)piperidine (2.2 equiv.) [from Step 1 of example 25] in dioxane (0.1 M) was added potassium carbonate (3.3 equiv.). The resulting mixture was heated in a sealed tube at 130° C. for 0.5 h. The title compound was then filtered over silica gel with 50% ethyl acetate in hexane.

### Step 5: 5-{3-[4-(2-Bromo-5-fluorophenoxy)piperidin-1-yl]-1,2,4-triazin-6-yl}nicotinic acid

The title compound was prepared as described in Step 4 of example 29, except that K₂HPO₄ was used in the work-up procedure instead of ammonium chloride.

MS: m/z 471.7 (ESI −).

### Example 34

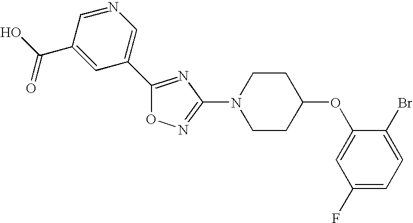

### 5-{3-[4-(2-Bromo-5-fluorophenoxy)piperidin-1-yl]-1 2,4-oxadiazol-5-yl}nicotinic acid

### Step 1: 4-(2-Bromo-5-fluorophenoxy)piperidine-1-carbonitrile

To a solution of 4-(2-bromo-5-fluorophenoxy)piperidine in THF (0.3 M) was added cyanogen bromide (1 equiv.) followed by triethylamine (1 equiv.) at 0° C. The mixture was warmed to RT and stirred for a further 1 h. The solvent was evaporated and the residue diluted with 1N HCl. The aqueous layer was extracted with EtOAc. The combined organic fractions were washed with water and dried over Na₂SO₄. The solvent was evaporated under reduced pressure to afford the title product as a solid which was used in the next step without further purification.

¹H NMR (500 MHz, acetone-d₆): δ 7.62 (dd, 1H), 7.08 (dd, 1H), 6.76 (td, 1H), 4.88-4.84 (m, 1H), 3.55-3.48 (m, 2H), 3.32-3.25 (m, 2H), 2.16-2.09 (m, 2H), 1.99-1.91 (m, 2H).

### Step 2: 4-(2-Bromo-5-fluorophenoxy)-N′-hydroxypiperidine-1-carboximidamide

A mixture of 4-(2-bromo-5-fluorophenoxy)piperidine-1-carbonitrile, hydroxylamine hydrochloride (3.0 equiv.) and Na₂CO₃ (17 equiv.) in 4:1 EtOH/water (0.2 M) was heated at 80° C. for 1 h. The solvent was evaporated, the residue was acidified with 6N HCl and washed with Et₂O . The aqueous layer was basified with solid Na₂CO₃ and extracted EtOAc. The combined organic fractions were dried over Na₂SO₄ and the solvent evaporated under reduced pressure to give the product as a foam which was used in the next step without further purification. MS: m/z 332, 334 (MH⁺).

### Step 3: Methyl 5-{3-[4-(2-bromo-5-fluorophenoxy)piperidin-1-yl]-1,2,4-oxadiazol-5-yl}nicotinate

A mixture of 5-(methoxycarbonyl)nicotinic acid in thionyl chloride (30 equiv.) was heated at 80° C. for 2 h. The excess thionyl chloride was evaporated. The residue was diluted with THF and then evaporated and dried under high vacuum. The mixture was dissolved in THF (0.5 M), 4-(2-bromo-5-fluorophenoxy)-N′-hydroxypiperidine-1-carboximidamide (1 equiv.) was added followed by triethylamine (3.0 equiv.). After 0.5 h, the mixture was heated at 80° C. for 1 h. The solvent was evaporated and saturated Na₂CO₃ was added. The aqueous layer was extracted EtOAc. The combined organic fractions were dried over Na₂SO₄ and the solvent was evaporated. Purification by Combiflash (SiO₂-12 g, gradient elution of 30-50% EtOAc/hexanes over 25 min) afforded the title product as a foam.

¹H NMR (500 MHz, acetone-d₆): δ 9.41-9.38 (m, 1H), 9.32-9.29 (m, 1H), 8.81 (s, 1H), 7.60 (dd, 1H), 7.10 (dd, 1H), 6.74 (td, 1H), 4.93-4.89 (m, 1H), 4.02 (s, 3H), 3.84-3.77 (m, 2H), 3.66-3.59 (m, 2H), 2.19-2.12 (m, 2H), 2.00-1.92 (m, 2H). MS: m/z 477, 479 (MH⁺).

### Step 4: 5-{3-[4-(2-Bromo-5-fluorophenoxy)piperidin-1-yl]-1,2,4-oxadiazol-5-yl}nicotinic acid

The title compound was prepared in the same manner as described in Step 4 of Example 22 from methyl 5-{3-[4-(2-bromo-5-fluorophenoxy)piperidin-1-yl]-1,2,4-oxadiazol-5-yl}nicotinate and 1 M aqueous NaOH.

¹H NMR (500 MHz, acetone-d₆): δ 9.44 (d, 1H), 9.37 (d, 1H), 8.89 (s, 1H), 7.63 (dd, 1H), 7.13 (dd, 1H), 6.76 (td, 1H), 4.94 (t, 1H), 3.87-3.80 (m, 2H), 3.68-3.62 (m, 2H), 2.19-2.12 (m, 2H), 2.00-1.93 (m, 2H). MS: m/z 462, 465 (MH⁺).

### Example 35

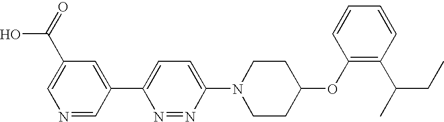

### 5-{6-[4-(2-sec-Butylphenoxy)piperidin-1-yl]pyridazin-3-yl}nicotinic acid

### Step 1: Ethyl 5-{6-[4-(2-sec-butylphenoxy)piperidin-1-yl]pyridazin-3-yl}nicotinate

The title compound was prepared, as described in Step 1 of example 21, using ethyl 5-[6-(4-hydroxypiperidin-1-yl)pyridazin-3-yl]nicotinate [from Step 2 of example 13] and 2-sec-butylphenol for the Mitsunobu reaction.

### Step 2: 5-{6-[4-(2-sec-Butylphenoxy)piperidin-1-yl]pyridazin-3-yl}nicotinic acid

The title compound was prepared from ethyl 5-{6-[4-(2-sec-butylphenoxy)piperidin-1-yl]pyridazin-3-yl}nicotinate as described in Step 4 of example 30, except that after ethyl acetate extraction the title compound was extracted with 1 M NaOH in ethyl acetate followed by the addition of 2 M HCl and extraction with ethyl acetate. MS: m/z 433.0 (ESI +).

### Example 36

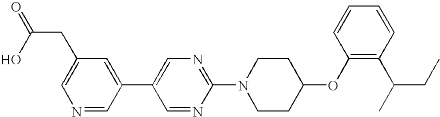

### (5-{2-[4-(2-sec-Butylphenoxy)piperidin-1-yl]pyrimidin-5-yl}pyridin-3-yl)acetic acid

### Step 1: 5-Bromo-2-[4-(2-sec-butylphenoxy)piperidin-1-yl]pyrimidine

The title compound was prepared, as described in Step 1 of example 21, from 1-(5-bromopyrimidin-2-yl)piperidin-4-ol from Step 1 of example 29 and 2-sec-butylphenol for the Mitsunobu reaction.

### Step 2: Methyl(5-{2-[4-(2-sec-butylphenoxy)piperidin-1-yl]pyrimidin-5-yl}pyridin-3-yl)acetate

The title compound was prepared, as described in Step 2 of example 29, using methyl [5-(4,4,5,5-tetramethyl-1,3,2-dioxaborolan-2-yl)pyridin-3-yl]acetate [from Step 1 of example 30] and 5-bromo-2-[4-(2-sec-butylphenoxy)piperidin-1-yl]pyrimidine.

### Step 3: (5-{2-[4-(2-sec-Butylphenoxy)piperidin-1-yl]pyrimidin-5-yl}pyridin-3-yl)acetic acid

The title compound was prepared, as described in Step 5 of example 33, using methyl(5-{2-[4-(2-sec-butylphenoxy)piperidin-1-yl]pyrimidin-5-yl}pyridin-3-yl)acetate. MS: m/z 447.3 (ESI +).

### Example 37

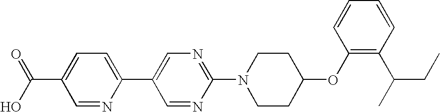

### 6-{2-[4-(2-sec-Butylphenoxy)piperidin-1-yl]pyrimidin-5-yl}nicotinic acid

### Step 1: 2-[4-(2-sec-Butylphenoxy)piperidin-1-yl]-5-(4,4,5,5-tetramethyl-1,3,2-dioxaborolan-2-yl)pyrimidine

The title compound was prepared, as described in Step 3 of example 1, from 5-bromo-2-[4-(2-sec-butylphenoxy)piperidin-1-yl]pyrimidine (example 36, step 1).

### Step 2: Methyl 6-{2-[4-(2-sec-butylphenoxy)piperidin-1-yl]pyrimidin-5-yl}nicotinate

The title compound was prepared, as described in Step 4 of example 1, using 2-[4-(2-sec-butylphenoxy)piperidin-1-yl]-5-(4,4,5,5-tetramethyl-1,3,2-dioxaborolan-2-yl)pyrimidine and methyl 6-bromonicotinate.

### Step 3: 6-{2-[4-(2-sec-Butylphenoxy)piperidin-1-yl]pyrimidin-5-yl}nicotinic acid

The title compound was prepared, as described in Step 5 of example 33, from methyl 6-{2-[4-(2-sec-butylphenoxy)piperidin-1-yl]pyrimidin-5-yl}nicotinate. MS: m/z 431.4 (ESI −).

### Example 38

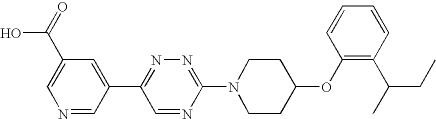

### 5-{3-[4-(2-sec-Butylphenoxy)piperidin-1-yl]-1,2,4-triazin-6-yl}nicotinic acid

### Step 1: tert-Butyl 4-(2-sec-butylphenoxy)piperidine-1-carboxylate

To a solution of tert-butyl 4-hydroxypiperidine-1-carboxylate in THF (0.1 M) at 0° C. were added 2-sec-butylphenol (1.1 equiv.), triphenylphosphine (1.0 equiv.), and DEAD (1.1 equiv.). After a period of 36 h, the reaction mixture was partitioned between ethyl acetate and 2 M sodium hydroxide. The organic phase was washed with brine, dried over MgSO₄, filtered and concentrated. The crude residue was purified by flash chromatography eluting with 20% ethyl acetate in hexane.

### Step 2: 4-(2-sec-Butylphenoxy)piperidine

To a solution of tert-butyl 4-(2-sec-butylphenoxy)piperidine-1-carboxylate in dichloromethane (0.2 M) at 0° C. was added TFA (5 equiv.). The resulting reaction mixture was then stirred at room temperature for 3 h. The TFA was removed under reduced pressure and the crude product was partitioned between ethyl acetate and 2 M sodium hydroxide. The organic phase was separated, dried over MgSO₄, filtered and evaporated. The title compound was purified by flash chromatography with NH₄OH/MeOH/CHCl₃ (1:9:90 to 1:14:85).

### Step 3: Ethyl 5-{3-[4-(2-sec-butylphenoxy)piperidin-1-yl]-1,2,4-triazin-6-yl}nicotinate

A mixture of ethyl 5-(3-bromo-1,2,4-triazin-6-yl)nicotinate [from Step 3 of example 33] and 4-(2-sec-butylphenoxy)piperidine (1.2 equiv.) in dioxane was added potassium carbonate (2.0 equiv.). The mixture was heated in the microwave at 130° C. for 30 min. The crude reaction mixture was then filtered and washed with dichloromethane and concentrated. The title compound was purified by flash chromatography eluting with 15% acetone in dichloromethane.

### Step 4: 5-{3-[4-(2-sec-Butylphenoxy)piperidin-1-yl]-1,2,4-triazin-6-yl}nicotinic acid

The title compound was prepared, as described in Step 4 of example 30, from ethyl 5-{3-[4-(2-sec-butylphenoxy)piperidin-1-yl]-1,2,4-triazin-6-yl}nicotinate.

¹H NMR (500 MHz, acetone-d₆): δ 9.44 (s, 1H), 9.20 (s, 1H), 9.00 (s, 1H), 8.95 (s, 1H), 7.25-6.95 (m, 3H), 4.85 (m, 1H), 4.35 (m, 2H), 4.00 (m, 4H), 2.20 (m, 2H), 1.95 (m, 3H), 1.65 (m, 2H), 1.20 (d, 3H), 0.85 (t, 3H).

### Example of a Pharmaceutical Formulation

As a specific embodiment of an oral composition of a compound of the present invention, 50 mg of the compound of any of the Examples is formulated with sufficient finely divided lactose to provide a total amount of 580 to 590 mg to fill a size O hard gelatin capsule.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. Likewise, the pharmacologic response observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended therefore that the invention be limited only by the scope of the claims which follow and that such claims be interpreted as broadly as is reasonable.

1. A compound of structural formula I: 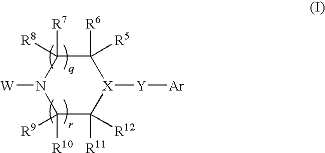
or a pharmaceutically acceptable salt thereof wherein
each m is independently an integer from 0 to 4;
each n is independently an integer from 0 to 2;
each s is independently an integer from 1 to 3;
each t is independently an integer from 1 to 3;
q is 0 or 1;
r is 0 or 1;
Z is 0, S, or NR⁴;
X—Y is N—C(O), N—CR^{a}R^{b}, CR¹⁴—O, CR¹⁴—S(O)₀₋₂, or CR¹³—CR^{a}R^{b};
W is heteroaryl selected from the group consisting of: 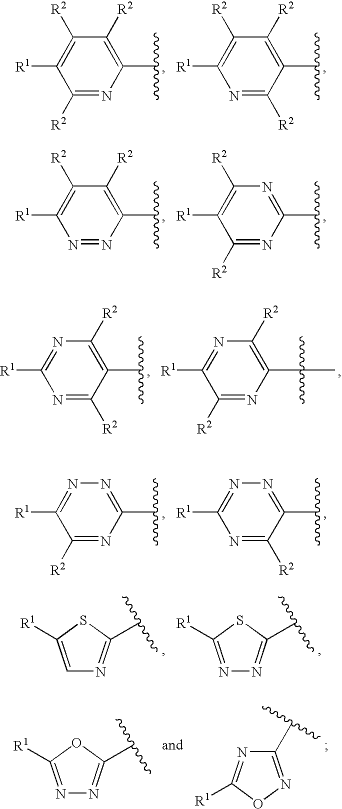
Ar is phenyl, naphthyl, or heteroaryl optionally substituted with one to five R³ substituents;
R^{a} and R^{b} are each independently hydrogen or C₁₋₃ alkyl, wherein alkyl is optionally substituted with one to three substituents independently selected from fluorine and hydroxy;
R¹ is heteroaryl selected from the group consisting of: 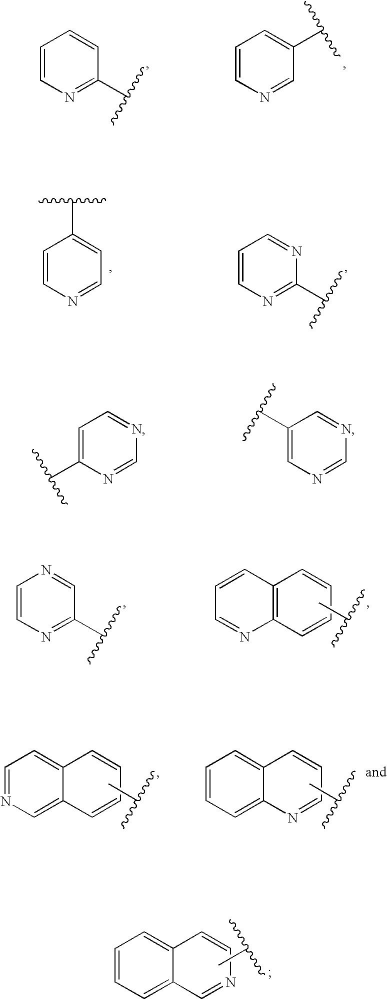
wherein heteroaryl is monosubstituted with —(CH₂)ₘCO₂H or —(CH₂)ₘCO₂C₁₋₃ alkyl and optionally substituted with one to three substituents independently selected from the group consisting of cyano, halogen, C₁₋₄ alkyl, C₁₋₄ alkoxy, C₁₋₄ alkylthio, C₁₋₄ alkylsulfonyl, and trifluoromethyl;
each R² is independently selected from the group consisting of:
hydrogen,
halogen,
hydroxy,
cyano,
amino,
nitro,
C₁₋₄ alkyl, optionally substituted with one to five fluorines,
C₁₋₄ alkoxy, optionally substituted with one to five fluorines,
C₁₋₄ alkylthio, optionally substituted with one to five fluorines,
C₁₋₄ alkylsulfonyl,
carboxy,
C₁₋₄ alkyloxycarbonyl, and
C₁₋₄ alkylcarbonyl;
each R³ is independently selected from the group consisting of:
C₁₋₆ alkyl,
C₂₋₆ alkenyl,
(CH₂)ₙ-phenyl,
(CH₂)ₙ-naphthyl,
(CH₂)ₙ-heteroaryl,
(CH₂)ₙ-heterocyclyl,
(CH₂)ₙC₃₋₇ cycloalkyl,
halogen,
nitro,
(CH₂)ₙOR⁴,
(CH₂)ₙN(R⁴)₂,
(CH₂)ₙC≡N,
(CH₂)ₙCO₂R⁴,
(CH₂)ₙNR⁴SO₂R⁴
(CH₂)ₙSO₂N(R⁴)₂,
(CH₂)ₙS(O)₀₋₂R⁴,
(CH₂)ₙNR⁴C(O)N(R⁴)₂,
(CH₂)ₙC(O)N(R⁴)₂,
(CH₂)ₙNR⁴C(O)R⁴,
(CH₂)ₙNR⁴CO₂R⁴,
(CH₂)ₙC(O)R⁴,
O(CH₂)ₙC(O)N(R⁴)₂,
(CH₂)ₛ-Z-(CH₂)ₜ-phenyl,
(CH₂)ₛ-Z-(CH₂)ₜ-naphthyl,
(CH₂)ₛ-Z-(CH₂)ₜ-heteroaryl,
(CH₂)ₛ-Z-(CH₂)ₜ-heterocyclyl,
(CH₂)ₛ-Z-(CH₂)ₜ—C₃₋₇ cycloalkyl,
(CH₂)ₛ-Z-(CH₂)ₜ—OR⁴,
(CH₂)ₛ-Z-(CH₂)ₜ—N(R⁴)₂,
(CH₂)ₛ-Z-(CH₂)ₜ—NR⁴SO₂R⁴,
(CH₂)ₛ-Z-(CH₂)ₜ—C≡N,
(CH₂)ₛ-Z-(CH₂)ₜ—CO₂R⁴,
(CH₂)ₛ-Z-(CH₂)ₜ—SO₂N(R⁴)₂,
(CH₂)ₛ-Z-(CH₂)ₜ—S(O)₀₋₂R⁴,
(CH₂)ₛ-Z-(CH₂)ₜ—NR⁴C(O)N(R⁴)₂,
(CH₂)ₛ-Z-(CH₂)ₜ—C(O)N(R⁴)₂,
(CH₂)ₛ-Z-(CH₂)ₜ—NR⁴C(O)R⁴,
(CH₂)ₛ-Z-(CH₂)ₜ—NR⁴CO₂R⁴,
(CH₂)ₛ-Z-(CH₂)ₜ—C(O)R⁴,
CF₃,
CH₂CF₃,
OCF₃, and
OCH₂CF₃;
in which phenyl, naphthyl, heteroaryl, cycloalkyl, and heterocyclyl are optionally substituted with one to three substituents independently selected from halogen, hydroxy, C₁₋₄ alkyl, trifluoromethyl, and C₁₋₄ alkoxy; and wherein any methylene (CH₂) carbon atom in R³ is optionally substituted with one to two groups independently selected from fluorine, hydroxy, and C₁₋₄ alkyl; or two substituents when on the same methylene (CH₂) group are taken together with the carbon atom to which they are attached to form a cyclopropyl group;
each R⁴ is independently selected from the group consisting of
hydrogen,
C₁₋₆ alkyl,
(CH₂)ₙ-phenyl,
(CH₂)ₙ-heteroaryl,
(CH₂)ₙ-naphthyl, and
(CH₂)ₙC₃₋₇ cycloalkyl;
wherein alkyl, phenyl, heteroaryl, and cycloalkyl are optionally substituted with one to three groups independently selected from halogen, C₁₋₄ alkyl, and C₁₋₄ alkoxy; or two R⁴ groups together with the atom to which they are attached form a 4- to 8-membered mono- or bicyclic ring system optionally containing an additional heteroatom selected from O, S, NH, and NC₁₋₄ alkyl;
R⁵, R⁶, R⁷, R⁸, R⁹, R¹⁰, R¹¹, and R¹² are each independently hydrogen, fluorine, or C₁₋₃ alkyl, wherein alkyl is optionally substituted with one to three substituents independently selected from fluorine and hydroxy;
R¹³ is hydrogen, C₁₋₃ alkyl, fluorine, or hydroxy; and
each R¹⁴ is hydrogen or C₁₋₃ alkyl.

2. The compound of claim 1 wherein m is 0 or 1.

3. The compound of claim 1 wherein q and r are both 1.

4. The compound of claim 1 wherein X—Y is CH—O.

5. The compound of claim 4 wherein Ar is phenyl substituted with one to three R³ substituents.

6. The compound of claim 1 wherein R⁵, R⁶, R⁷, R⁸, R⁹, R¹⁰, R¹¹, and R¹² are each hydrogen.

7. The compound of claim 1 wherein W is heteroaryl selected from the group consisting of:

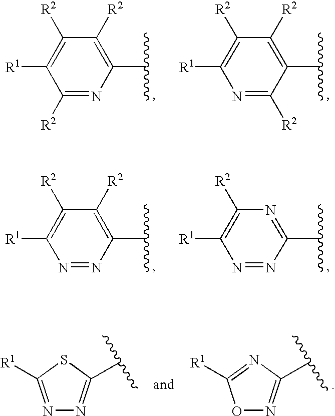

8. The compound of claim 1 wherein each R² is hydrogen.

9. The compound of claim 1 wherein R¹ is pyridin-3-yl or pyrimidin-2-yl, wherein R¹ is monosubstituted with a substituent selected from the group consisting of:
—CO₂H,
—CH₂CO₂H,
—CO₂C₁₋₃ alkyl, and
—CH₂CO₂C₁₋₃ alkyl;
and optionally substituted with one to two substituents independently selected from the group consisting of cyano, halogen, C₁₋₄ alkyl, C₁₋₄ alkoxy, C₁₋₄ alkylthio, C₁₋₄ alkylsulfonyl, and trifluoromethyl.

10. The compound of claim 9 wherein R¹ is selected from the group consisting of: 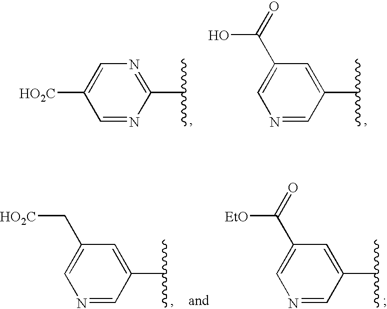
wherein R¹ is optionally substituted with one to two substituents independently selected from the group consisting of halogen, C₁₋₄ alkyl, and trifluoromethyl.

11. The compound of claim 1 wherein q and r are both 0; X—Y is CH—O; W is heteroaryl selected from the group consisting of: 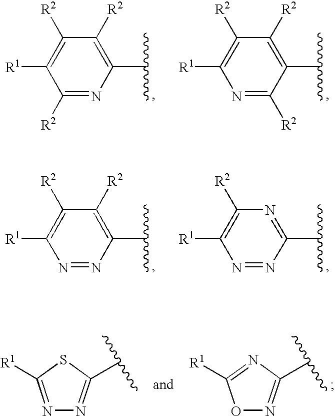
and R¹ is pyridin-3-yl or pyrimidin-2-yl, wherein R¹ is monosubstituted with a substituent selected from the group consisting of:
—CO₂H,
—CH₂CO₂H,
—CO₂C₁₋₃ alkyl, and
—CH₂CO₂C₁₋₃ alkyl;
and optionally substituted with one to two substituents independently selected from the group consisting of cyano, halogen, C₁₋₄ alkyl, C₁₋₄ alkoxy, C₁₋₄ alkylthio, C₁₋₄ alkylsulfonyl, and trifluoromethyl.

12. The compound of claim 11 wherein R², R⁵, R⁶, R⁷, R⁸, R⁹, R¹⁰, R¹¹, and R¹² are each hydrogen.

13. A compound which is selected from the group consisting of: 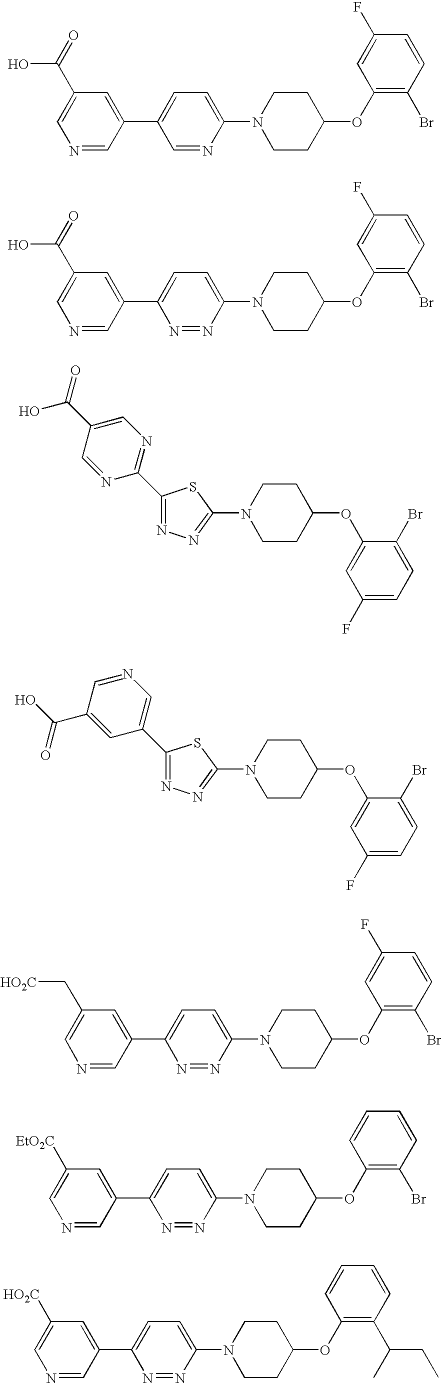 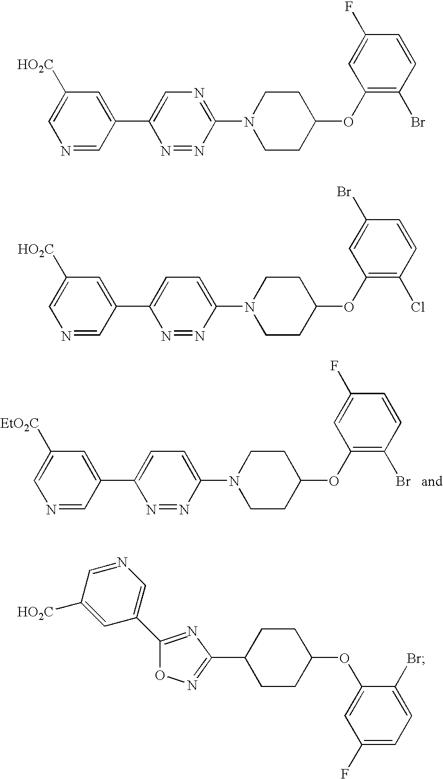
or a pharmaceutically acceptable salt thereof.

14. A pharmaceutical composition comprising a compound in accordance with claim 1 in combination with a pharmaceutically acceptable carrier.

15-18. (canceled)

19. A method for treating non-insulin dependent (Type 2) diabetes, insulin resistance, hyperglycemia, a lipid disorder, obesity, and fatty liver disease in a mammal in need thereof which comprises the administration to the mammal of a therapeutically effective amount of a compound of claim 1.

20. The method of claim 19 wherein said lipid disorder is selected from the group consisting of dyslipidemia, hyperlipidemia, hypertriglyceridemia, atherosclerosis, hypercholesterolemia, low HDL, and high LDL.
